(19) United States
(12) Patent Application Publication
(10) Pub. No.: US 2012/0122949 A1
(43) Pub. Date: May 17, 2012

(54) ULTRA-HIGH STRENGTH INJECTABLE HYDROGEL AND PROCESS FOR PRODUCING THE SAME

(75) Inventors: Yuichi Tei, Tokyo (JP); Takamasa Sakai, Tokyo (JP); Nobuo Sasaki, Tokyo (JP); Mitsuhiro Shibayama, Tokyo (JP); Shigeki Suzuki, Tokyo (JP)

(73) Assignees: THE UNIVERSITY OF TOKYO, Bunkyo-ku, Tokyo (JP); NEXT21 K.K., Bunkyo-ku, Tokyo (JP)

(21) Appl. No.: 13/139,629

(22) PCT Filed: Jun. 19, 2009
(86) PCT No.: PCT/JP2009/002789

§ 371 Date: Sep. 22, 2011

(30) Foreign Application Priority Data
Dec. 19, 2008 (JP) 2008-324313

(51) Int. Cl. A61K 31/132; A61P 19/00; A61K 31/4025

(52) U.S. Cl. 514/422; 514/668

(57) ABSTRACT

[Problem]

The present invention is intended to provide high-strength hydrogels and method for fabricating the same. The present invention is intended to provide the method for fabricating the hydrogels with different decomposition rates.

[Solution to Problem] The present invention is based on a knowledge that high-strength hydrogels can be fabricated by controlling pH of solution, ionic strength in the solution, and buffer concentration in the solution. In addition, the present invention is based on a knowledge that the high-strength hydrogels which have homogeneous macromolecular network structure can be fabricated by polymerizing four-branching compounds after having dispersed the four-branching compounds homogeneously.

### TECHNICAL FIELD

The present invention relates to hydro-gels of three-dimensional network structure and method for fabricating the same.

### TECHNICAL BACKGROUND

Gels with polymer have been conventionally used in medical purpose such as sealing and prevention of adhesion. Gels fabricated by mixing many branched polymers is disclosed in JP 2000-502,380 official gazette. However, the gels provided by the official gazette is weak in strength and cannot apply to load sites in a living body such as knee cartilage, vertebral body, or intervertebral disk.

In an international publication pamphlet WO2006/013612, a method for fabricating hydrogels by mixing two types of monomers is disclosed. In the pamphlet, the hydrogels are fabricated by mixing two types of monomers to form multiplex network structure. However, the hydrogels disclosed in the pamphlet are not strong enough to be applicable to the load sites in a living body.

In this way, since the strength of the gels used for operation on knee cartilage and intervertebral disk (nucleus pulposus) is not enough, degeneration of the gels occurs when introduced and used in a living body for a long term. Therefore, it was a problem that period operation is required when they are used at a weight-bearing point.

### PRIOR ART REFERENCE

### Patent Literatures

Patent literature 1: JP 2000-502,380 Patent Gazette.

Patent literature 2: international publication Pamphlet WO2006/013612

### SUMMARY OF INVENTION

### Problem to be Solved by the Invention

The present invention aims to provide high-strength hydrogels and method for fabricating the same.

The present invention aims to provide method for fabricating hydrogels with different decomposition rates.

### Means for Solving Problem

The present invention is based on a knowledge that high-strength hydrogels can be fabricated by adjusting pH, ionic strength, and buffer concentration of solution. In addition, the present invention is based on a knowledge that high-strength hydrogels that have homogeneous macromolecular network structure can be fabricated by polymerizing two types of four-branching compounds after having dispersed homogeneously the two types of the four-branching compounds.

A first aspect of the present invention relates to a method for fabricating the hydrogels. The method for manufacturing the hydrogels in the present invention comprises a step of mixing a first solution, which comprises a first four-branching compound and a first buffer solution, and a second solution, which comprises a second four-branching compound and a second buffer solution. The said first four-branching compound is expressed in the following chemical formula (I).

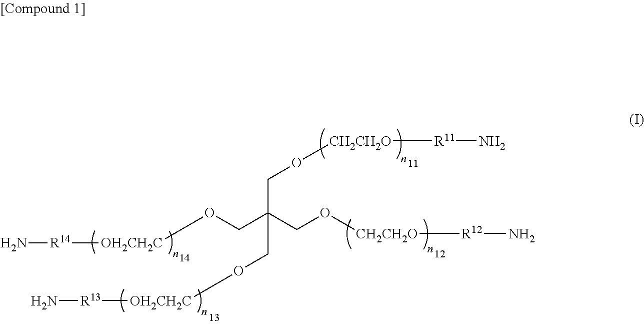

In the said chemical formula [I], n₁₁ to n₁₄ are, each may be the same or different, an integer that is any one of 25 to 250. In the chemical formula (I), R₁₁ to R₁₄ are, each may be the same or different, C₁-C₇ alkylene group, C₂-C₇ alkenylene group, —NH—R¹⁵—, —CO—R¹⁵—, —R¹⁶—O—R¹⁷—, —R¹⁶—NH—R¹⁷—, —R¹⁶—CO₂R¹⁷—, —R¹⁶—CO₂—NH—R¹⁷—, —R¹⁶—CO—R¹⁷—, or —R¹⁶—CO—NH—R¹⁷—, wherein R¹⁵ is C₁-C₇ alkylene group, R¹⁶ is C₁-C₃ alkylene group, and R¹⁷ is C₁-C₅ alkylene group.

The said second four-branching compound is expressed in the following chemical formula (II).

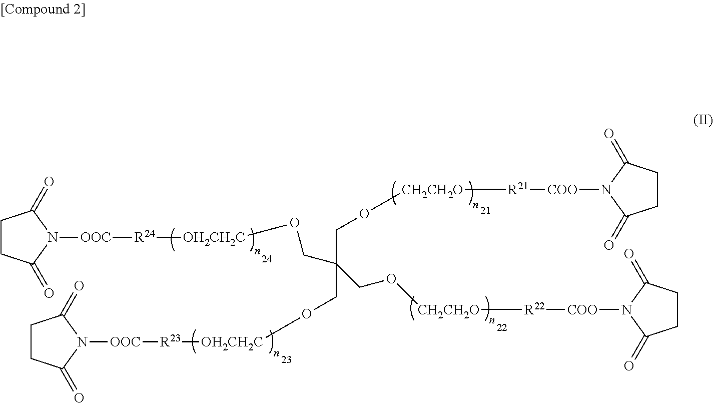

In the said chemical formula (II), n₂₁ to n₂₄ are, each may be the same or different, an integer that is any one of 20 to 250. In the chemical formula (II), R²¹ to R²⁴ are, each may be the same or different, C₁-C₇ alkylene group, C₂-C₇ alkenylene group, —NH—R²⁵—, —CO—R²⁵—, —R²⁶—O—R²⁷—, —R²⁶—NH—R²⁷—, —R²⁶—CO₂—R²⁷—, —R²⁶—CO₂—NH—R¹⁷—, —R²⁶—CO—R²⁷—, or —R²⁶—CO—NH—R²⁷, wherein R²⁵ is C₁-C₇ alkylene group, R²⁶ is C₁-C₃ alkylene group, R²⁷ is C₁-C₅ alkylene group.

In addition, pH of the first buffer solution is from 5 to 9, and concentration of the said first buffer is from 20 to 200 mM, and pH of the said second buffer solution is from 5 to 9, and concentration of the said second buffer solution is from 20 to 200 mM. Furthermore, the pH of the first solution is higher than the said pH of the second solution. Reactions as shown in FIGS. 1 and 2 can take place by using such two types of the four-branching compounds, and then hydrogels with homogeneous network structure can be fabricated.

As shown above, the first four-branching compound of the present invention has amino groups. In acid solution, the amino groups of the first four-branching compound are easy to turn into cationic state and tend to repel each other (FIGS. 2 and 3A). Then the cationic amino groups decrease the reactivity with functional group (N-hydroxy-succinimidyl (NHS)) of the second four-branching compound (FIG. 2). On the other hand, the reactivity with the second four-branching compound increases when the pH of the first solution becomes high (shifts to alkaline side), because the amino groups of the first four-branching compound become easy to change from —NH₃⁺ to —NH₂ (FIG. 2). However, when the pH of the solution is greater than or equal to 7, the ester linkage in the second four-branching compound becomes easy to be broken, and the reactivity with the first four-branching compound decreases. Therefore gel strength becomes weak. Therefore the pH of the first and second solutions can be adjusted by adjusting the pH of the first and second buffer solutions, and then the reaction rate of the first and the second four-branching compounds can be adjusted and high-strength hydrogels can be fabricated.

In addition, as shown in the following embodiment, when concentration of buffer solution is too low, pH buffer capacity of the solution decreases and then the high-strength hydrogels cannot be fabricated. In addition, if the buffer concentration is too high, the high-strength hydrogels cannot be fabricated, because the high buffer concentration impedes mixing of the first and second four-branching compounds. Therefore, as shown in the following embodiment, the high-strength hydrogels that have homogeneous structure can be fabricated by setting the concentration of the buffer within the range of 20 mM to 200 mM.

Therefore, the time for gelation of the hydrogels (reaction rate) can be adjusted by adjusting, as mentioned above, the pH of the first and second buffer solutions and the buffer concentration in solution, and furthermore the high-strength hydrogels with homogeneous structure can be fabricated.

In the first aspect of a preferred embodiment of the present invention, the said first buffer solution comprises one or more of phosphate buffer or phosphate buffered saline. The said second buffer solution comprises one or more of the phosphate buffer, citric acid/phosphate buffer, the phosphate buffered saline, or citric acid/phosphate buffered saline. By using such buffers as shown in the following embodiment, the high-strength hydrogels with homogeneous structure can be fabricated.

In the first aspect of a preferred embodiment of the present invention, salt concentration of the mixed solution after the said mixing process is 0 to 1×10² mM, and preferably may be 1×10⁻¹ to 1×10² mM. If the salt concentration in the mixed solution is high, anion of the salt interacts with cation of the first four-branching compound, which results in reduction of repulsion between the cations. When the repulsion between the cations decreases, the two types of the four-branching compounds become hard to be mixed homogeneously (FIGS. 3A and 3B). If the two types of the four-branching compounds are not mixed homogeneously, the hydrogels with homogeneous three-dimensional structure cannot be fabricated, and the strength of the hydrogels becomes weak. As shown in the following embodiment, when the salt concentration in the mixed solution rises, the strength of the gels becomes weak. Therefore, as shown in the following embodiment, by setting the salt concentration to the above-mentioned concentration, the two types of the four-branching compounds are mixed homogeneously without influence of anion of the salt and then the high-strength hydrogels can be fabricated.

In the first aspect of a preferred embodiment of the present invention, the pH of the said first buffer solution is from 5 to 9 and the concentration of the said first buffer solution is phosphate buffer of 20 to 100 mM. In the said second solution, the said pH is 5 to 7.5, and the said second buffer solution is either of the phosphate buffer of 20 mM to 100 mM or the citric acid/phosphate buffer of 20 mM to 100 mM. As mentioned above, if the pH of the first solution is high, the first and the second four-branching compounds are hard to be mixed homogeneously. In addition, if the pH of the second solution is too high, ester of the second four-branching compound is decomposed. When the ester of the second four-branching compound is decomposed, the terminal functional group of the four-branching compound is released. Thereby, the first four-branching compound cannot be bonded with the second four-branching compound. Therefore, the strength of the fabricated hydrogels decreases. Thus, as shown in the present invention, by setting the pH of the first solution to be 5 to 9 and the pH of the second solution to be 5 to 7.5, the first and the second four-branching compounds can be mixed efficiently and homogeneously, and the hydrogels with homogeneous three-dimensional structure can be fabricated. In addition, as shown in the following embodiment, if the buffer concentration is too low, the pH buffer capacity in the mixed solution is low. On the other hand, if the concentration is too high, the strength of the hydrogels decreases. Therefore more high-strength hydrogels can be effectively fabricated by setting the concentration of the first and the second buffers within the range of 20 to 100 mM.

In the first aspect of a preferred embodiment of the present invention, in the mixed solution after the said mixing process, average pH values from immediately after mixing to 30 seconds later are 6 to 8. As mentioned above, the first four-branching compound of the present invention has amino groups. The amino groups, more than or equal to 95% of which is at cationic state in solution with pH of less than or equal to 8, repel each other (FIG. 3A). In addition, the cationic amino group does not react with functional group (N-hydroxy-succinimidyl (NHS) group) of the second four-branching compound (FIG. 2). For this, by holding the pH of the mixed solution at 30 seconds after mixing to the range of 6 to 8, the first and the second four-branching compounds are prevented from bonding locally, and both the compounds can be homogeneously dispersed in solution (FIG. 3A). Then, as non-cationic amino groups (—NH₂) that are around 5% react with the NHS, equilibrium state of the amino groups of the first four-branching compound changes from —NH₃⁺ to —NH₂, and the reaction with the second four-branching compound progresses (FIG. 2). In this way, by keeping the fraction of the non-cationic amino group which can react with NHS to be around 5% by adjusting the pH of the solution after mixing the solutions, the first and the second four-branching compounds can be effectively prevented from being inhomogeneously mixed, which results in the increase in final reaction yield, and then homogeneous high-strength hydrogels can be fabricated.

The second aspect of the present invention relates to hydrogels fabricated with fabrication method comprising a step of mixing a first solution, which comprises a first four-branching compound and a first buffer solution, and a second solution, which comprises a second four-branching compound and a second buffer solution, to obtain a mixed solution. The said first four-branching compound is shown as the following chemical formula (I).

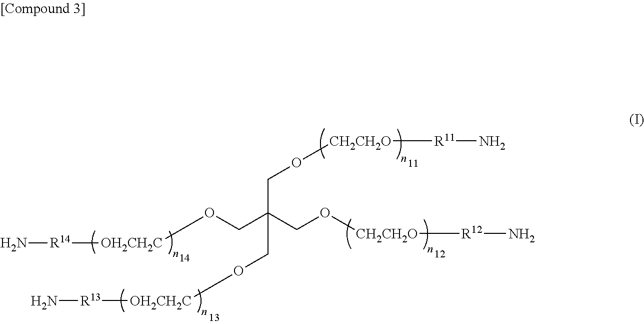

In the said chemical formula (I), n₁₁ to n₁₄ are, each may be the same or different, an integer that is any one of 25 to 250. In the formula (I), R¹¹ to R¹⁴ are, each may be the same or different, C₁-C₇ alkylene group, C₂-C₇ alkenylene group, —NH—R¹⁵—, —CO—R¹⁵—, —R¹⁶—O—R¹⁷—, —R¹⁶—NH—R¹⁷—, —R¹⁶—CO₂—R¹⁷—, —R¹⁶—CO₂—NH—R¹⁷—, —R¹⁶—CO—R¹⁷—, or —R¹⁶—CO—NH—R¹⁷—, wherein R¹⁵ is C₁-C₇ alkylene group, R¹⁶ is C₁-C₃ alkylene group, and R¹⁷ is C₁-C₅ alkylene group. The said second four-branching compound is shown as the following chemical formula (II).

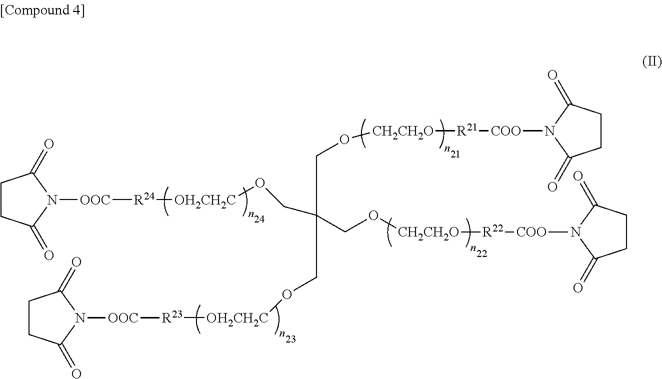

In the said chemical formula (II), n₂₁ to n₂₄ are, each may be the same or different, an integer that is any one of 20 to 250. In the chemical formula (II), R²¹ to R²⁴ are, each may be the same or different, C₁-C₇ alkylene group, C₂-C₇ alkenylene group, —NH—R²⁵—, —CO—R²⁵—, —R²⁶—O—R²⁷—, —R²⁶—NH—R²⁷—, —R²⁶—CO₂—NH—R²⁷—, —R²⁶—CO₂—NH—R¹⁷—, —R²⁶—CO—R²⁷—, or —R²⁶—CO—NH—R²⁷, wherein R²⁵ is C₁-C₇ alkylene group, R²⁶ is C₁-C₃ alkylene group, R²⁷ is C₁-C₅ alkylene group.

pH of the said first buffer solution is from 5 to 9 and concentration of the said first buffer solution is from 20 to 200 mM, and pH of the said second buffer solution is from 5 to 9 and concentration of the said second buffer solution is from 20 to 200 mM. The pH of the said first solution is preferably higher than the pH of the said second solution.

As shown in the following embodiment, the hydrogels fabricated using the fabrication method of the present invention have the strength to exceed that of cartilage in living body. In addition, as shown in the following embodiment, the hydrogels of the present invention do not exhibit cytotoxicity. Therefore, the hydrogels of the present invention can be effectively used for treatment of the defect of bones, cartilage or intervertebral disk, or of degeneration of the bones, the cartilage, or the intervertebral disk.

The third aspect of the present invention relates to hydrogels comprising a first four-branching compound and a second four-branching compound, wherein composition ratio of the first and the second four-branching compounds is 0.8:1 to 1.2:1. The said first four-branching compound is shown as the following chemical formula (I).

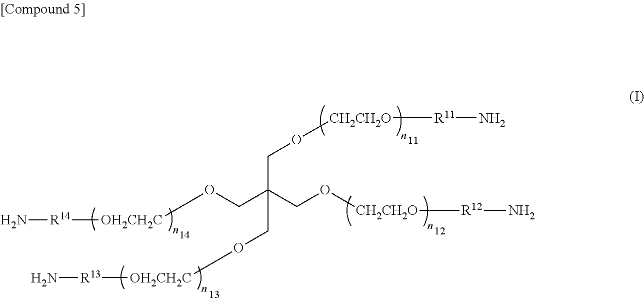

In the said chemical formula (I), n₁₁ to n₁₄ are, each may be the same or different, an integer that is any one of 25 to 250. In the said chemical formula (I), R¹¹ to R¹⁴ are, each may be the same or different, C₁-C₇ alkylene group, C₂-C₇ alkenylene group, —NH—R¹⁵—, —CO—R¹⁵—, —R¹⁶—O—R¹⁷—, —R¹⁶—NH—R¹⁷—, —R¹⁶—CO₂—R¹⁷—, —R¹⁶—CO₂—NH—R¹⁷—, —R¹⁶—CO—R¹⁷—, or —R¹⁶—CO—NH—R¹⁷—, wherein R¹⁵ is C₁-C₇ alkylene group, R¹⁶ is C₁-C₃ alkylene group, and R¹⁷ is C₁-C₅ alkylene group. The said second four-branching compound is shown as the following chemical formula (II).

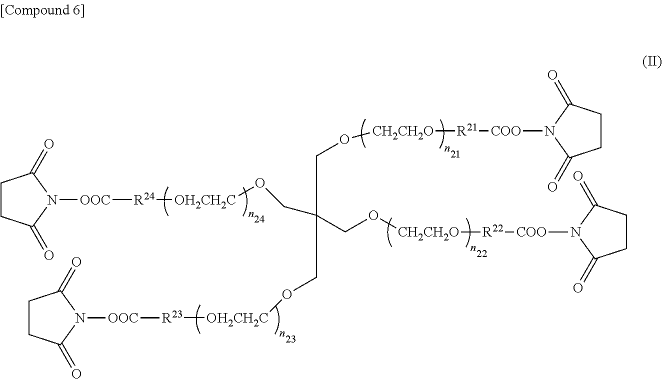

In the said chemical formula (II), n₂₁ to n₂₄ are, each may be the same or different, an integer that is any one of 20 to 250. R²¹ to R²⁴ are, each may be the same or different, C₁-C₇ alkylene group, C₂-C₇ alkenylene group, —NH—R₂₅—, —CO—R²⁵—, —R²⁶—O—R²⁷—, —R²⁶—NH—R²⁷—, —R²⁶—CO₂—R²⁷—, —R²⁶—CO₂—NH—R¹⁷—, —R²⁶—CO—R²⁷—, or —R²⁶—CO—NH—R²⁷, wherein R²⁵ is C₁-C₇ alkylene group, R²⁶ is C₁-C₃ alkylene group, R²⁷ is C₁-C₅ alkylene group.

The neutron scattering curve of the said hydrogels can be fitted by Orstein-Zernike function. As shown in the following embodiment, the scattering curve obtained from a group of the neutron scattering values measured for the hydrogels of the present invention is fitted by the curve expressed with OZ function. In other words, the hydrogels of the present invention have homogeneous gel structure. Having such homogeneous gel structure, the hydrogels become high-strength and can be suitably used in living body parts, which are subject to weight-bearing, such as knee cartilage, vertebra body, and intervertebral disk.

The third aspect of a preferred embodiment of the present invention is the hydrogels described in the above that compression breaking strength is 10 to 120 MPa. As shown in the following embodiment, the hydrogels of the present invention have the strength to exceed the strength of cartilage in living body (10 MPa). Therefore, they can be suitably used in living body parts, which are subject to weight-bearing, such as knee cartilage and vertebra body.

The fourth aspect of the present invention relates to hydrogels which comprise a first four-branching compound, a second four-branching compound and a third four-branching compound, wherein composition ratio of the first four-branching compound, the second four-branching compound, and the third four-branching compound is 0.3-0.7:0-0.65:0-0.65. The hydrogels of the present invention may comprise a first four-branching compound, a second four-branching compound and a third four-branching compound, wherein the composition ratio of the first four-branching compound. the second four-branching compound, and the third four-branching compound may be 0.3-0.7:0.1-0.65:0.1-0.65. The said first four-branching compound is expressed as the said chemical formula (I). In the said chemical formula (I), n₁₁ to n₁₄ are, each may be the same or different, an integer that is any one of 50 to 60, R¹¹ to R¹⁴ are, each may be the same or different, C₁-C₇ alkylene group. The said second four-branching compound is expressed as the said chemical formula (II). In the said chemical formula (II), n₂₁ to n₂₄ are, each may be the same or different, an integer that is any one of 45 to 55, R²¹ to R²⁴ are, each may be the same or different, —CO—R²⁵— and R²⁵ is C₁-C₇ alkylene group. The said third four-branching compound is expressed as the said chemical formula (II). In the said chemical formula (II), n₂₁ to n₂₄ are, each may be the same or different, an integer that is any one of 45 to 55, R²¹ to R²⁴ are, each may be the same or different, C₁-C₇ alkylene group. As shown in the following embodiment, decomposition rate can be adjusted by setting the hydrogels to such composition ratio, while retaining high-strength. Therefore, the hydrogels of the present invention can be decomposed to the reproduction rate in the parts where the hydrogels were introduced into, by adjusting the decomposition rate. Therefore, the hydrogels of the present invention can be suitably used for the treatment of the defect of bones, cartilage or intervertebral disk, or of degeneration of the bones, the cartilage, or the intervertebral disk.

### Advantageous Effect of the Invention

According to the present invention, high-strength hydrogels and method for fabricating the same can be provided.

The present invention can provide the hydrogels with different decomposition rates.

### BRIEF DESCRIPTION OF DRAWINGS

FIG. 1 illustrates the structure of the hydrogel.

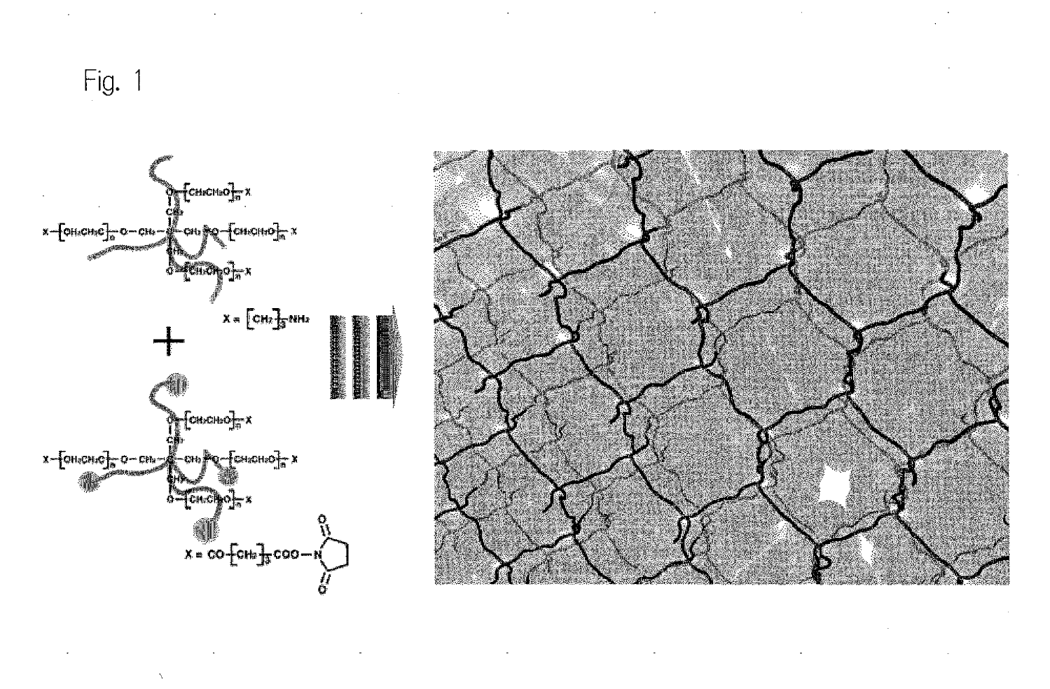

FIG. 2 illustrates the state of reaction of the first and second four-branching compounds.

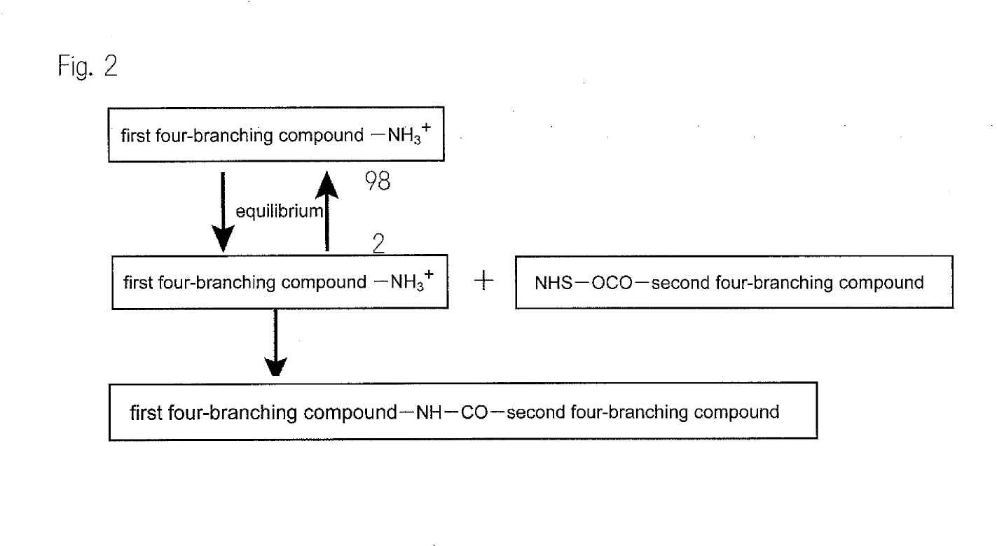

FIG. 3 illustrates schematically the distribution of the first and the second four-branching compounds in solution. FIG. 3A illustrates state that the first and the second four-branching compounds mix homogeneously in solution. FIG. 3B illustrates that distribution of the first and second four-branching compounds becomes inhomogeneous in solution by salt anion.

FIG. 4 illustrates a graph indicating compressive elastic modulus (kPa) of the gels in which TAPEG and TNPEG are mixed in the range of mole fraction (r) of 0.33 to 3.0.

FIG. 5 illustrates a graph indicating that breaking strain (%) and breaking strength (MPa) of the gels in which TAPEG and TNPEG are mixed in the range of mole fraction of 0.6 to 1.4.

FIG. 6 illustrates a graph indicating result of compression breaking strength measurement of the hydrogels.

FIG. 7 illustrates a graph indicating neutron scattering result of measurement of the hydrogels.

FIG. 8 illustrates a photograph of the hydrogels implanted in mouse back.

FIG. 9 illustrates a photograph of the hydrogels implanted in dog knee cartilage. FIGS. 9A to 9C illustrate photographs of the implanted parts at two months later after surgery. FIGS. 9D to 9F illustrate photographs of the implanted part at four months later after surgery.

FIG. 10 illustrates photographs of swine intervertebral disk where the hydrogels were implanted. FIG. 10A illustrates a photograph of hydrogel implantation in progress. FIG. 10B illustrates a photograph of intervertebral disk after implantation of the hydrogels.

FIG. 11 illustrates decomposition rate of the gels.

FIG. 12 illustrates cell proliferation activity in each cell of NIH3T3,MC3T3-E1, and ATDC5 in the presence of the hydrogels. In FIG. 12, the vertical axis shows the proliferative activity of the cell (absorbance level). FIG. 12A shows result of the proliferative activity of the NIH3T3 cell. FIG. 12B shows result of the proliferative activity of the MC3T3-E1 cell. FIG. 12C shows result of the proliferative activity of the ATDC5 cell.

### MODE FOR CARRYING OUT THE CLAIMED INVENTION

The first aspect of the present invention relates to method for fabricating hydrogels. The method for fabricating the hydrogels in the present invention comprises a step of mixing a first solution, which comprises a first four-branching compound and a first buffer solution, and a second solution, which comprises a second four-branching compound and a second buffer solution, to obtain a mixed solution.

The hydrogels are gelatinous material comprising hydrophilic macromolecule including a large quantity of water. The hydrogels of the present invention are made from more than two types of four-branching compounds.

A compound as expressed in the following chemical formula (I) is realized as the first four-branching compound of the present invention.

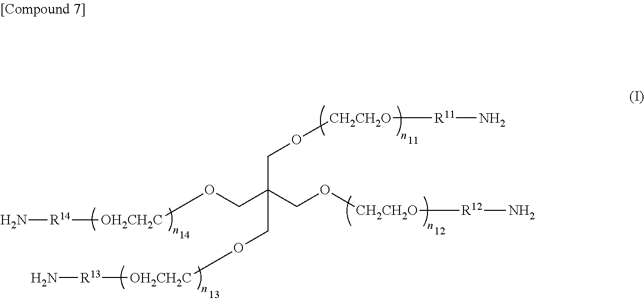

In the chemical formula (I), R¹¹ to R¹⁴ are, each may be the same or different, C₁-C₇ alkylene group, C₂-C₇ alkenylene group, —NH—R¹⁵—, —CO—R¹⁵—, —R¹⁶—O—R¹⁷—, —R¹⁶—NH—R¹⁷—, —R¹⁶—CO₂—R¹⁷—, —R¹⁶—CO₂—NH—R¹⁷—, —R¹⁶—CO—R¹⁷—, or —R¹⁶—CO—NH—R¹⁷—, wherein R¹⁵ is C₁-C₇ alkylene group, R¹⁶ is C₁-C₃ alkylene group, and R¹⁷ is C₁-C₅ alkylene group.

Each of n₁₁ to n₁₄ may be the same or different. If values of n₁₁ to n₁₄ are nearer to each other, the hydrogels can have more homogeneous conformation, which results in high strength. For this, it is preferable that these values are the same to obtain the high-strength hydrogels. If values of n₁₁ to n₁₄ are too high, strength of the hydrogels become weak, and if values of n₁₁ to n₁₄ are too low, the hydrogels become hard to be formed owing to steric hindrance of the compounds. Therefore, n₁₁ to n₁₄ are an integer that is any one of 25 to 250, preferably any one of 35 to 180, more preferably any one of 50 to 115, and highly preferably any one of 50 to 60. In addition, molecular weight of the first four-branching compound of the present invention is 5×10³ to 5×10⁴ Da, preferably 7.5×10³ to 3×10⁴ Da, and more preferably 1×10⁴ to 2×10⁴ Da.

In the above chemical formula (I), R¹¹-R¹⁴ is a linker region to tie the core moiety of the first four-branching compound to functional groups. Each of R¹¹ to R¹⁴ may be the same or different, but is preferably the same to fabricate high-strength hydrogels with homogeneous conformation. R¹¹ to R¹⁴ are C₁-C₇ alkylene group, C₂-C₇ alkenylene group, —NH—R¹⁵—, —CO—R¹⁵—, —R¹⁶—O—R¹⁷—, —R¹⁶—NH—R¹⁷—, —R¹⁶CO₂—R¹⁷—, —R¹⁶CO₂—NH—R¹⁷—, —R¹⁶—CO—R¹⁷—, or —R¹⁶—CO—NH—R¹⁷—, wherein R¹⁵ is C₁-C₇ alkylene group, R¹⁶ is C₁-C₃ alkylene group, and R¹⁷ is C₁-C₅ alkylene group.

Here the C₁-C₇ alkylene group means the alkylene group that the number of the carbon atom which may have branching is more than 1 and less than 7, and means linear C₁-C₇ alkylene group or C₂-C₇ alkylene group where the number of the carbon atoms including branching is more than 2 and less than 7. The examples of the C₁-C₇ alkylene group are a methylene group, an ethylene group, a propylene group, butylene group. The examples of the C₁-C₇ alkylene group are —CH₂—, —(CH₂)₂—, —(CH₂)₃—, —CH(CH₃)—, —(CH₂)₃—, —(CH(CH₃))₂—, —(CH₂)₂—CH(CH₃)—, —(CH₂)₃—CH(CH₃)—, —(CH₂)₂—CH(C₂H₅)—, —(CH₂)₆—, —(CH₂)₂—C(C₂H₅)₂—, and —(CH₂)₃C(CH₃)₂CH₂—.

The “C₂-C₇ alkenylene group” is the alkenylene group that has one or more of double bonds in chain or branched chain consisting of 2 to 7 carbon atoms, and the example is a bivalent group with the double bond that is formed by removing 2 to 5 hydrogen atoms adjacent to each other from the said alkylene group.

In addition, when a bond between linker moiety and core moiety of the first four-branching compound is an ester linkage, the first four-branching compound is easy to be decomposed in vivo. In contrast, when the bond between the linker moiety and the core moiety of the first four-branching compound is an ether linkage, the first four-branching compound is hard to be decomposed in vivo. In other words, decomposition properties of the first four-branching compound depend on types of R¹¹ to R¹⁴. Therefore, decomposition rate of the hydrogels fabricated can be also controlled by using the first four-branching compound. If the hydrogels which controlled the decomposition rate is fabricated, two or more than two types of the compounds of the chemical formula (I) expressed in the above may be also used. The C₁-C₇ alkylene group is preferable as R¹¹-R¹⁴ that forms the ether linkage, and ethylene group, propylene group, and butylene group are preferable.

In addition, as showed in the above chemical formula (I), the desired functional group of the first four-branching compound of the present invention is amino group. However, the hydrogels of the present invention have high-strength conformation by bonding the functional group of the first four-branching compound with nucleophilicity and the functional group of the second four-branching compound with electrophilicity by chemical reaction. Therefore, nucleophilic functional groups except the amino group can be used as a functional group of the first four-branching compound of the present invention. The —SH or —CO₂PhNO₂, where Ph indicates o-, m-, or p-phenylene group, can be cited as an example of such nucleophilic functional groups and well-known nucleophilic functional groups can be used appropriately by person skilled in art.

The first concentration of the first four-branching compound, as expressed in the above chemical formula (I), in solution, may be 10 mg/mL to 500 mg/mL. When concentration of the four-branching compound is too low, strength of the gels become weak, and when the concentration of the four-branching compound is too high, structure of the hydrogels becomes inhomogeneous and as a result the strength of the gels becomes weak. Therefore 20 to 400 mg/mL are preferable, and 50 mg/mL to 300 mg/mL are more preferable, and 100 to 200 mg/mL are further more preferable.

The compound expressed in the following chemical formula (II) is cited as an example of the second four-branching compound of the present invention.

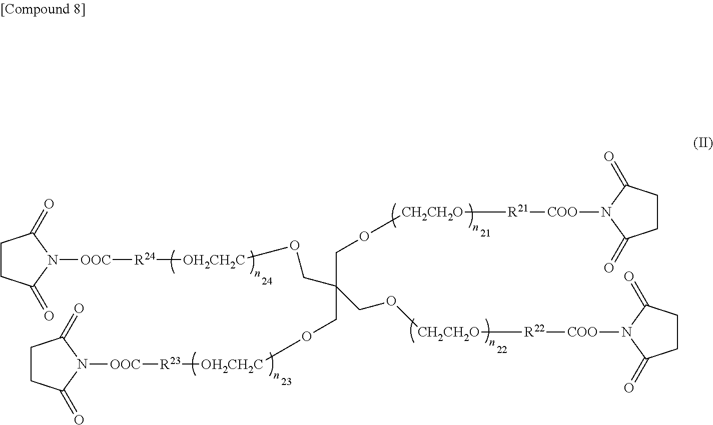

In the said chemical formula (II), n₂₁ to n₂₄ may be the same or different. If the values of n₂₁ to n₂₄ are near to each other, the hydrogels can have more homogeneous conformation, which preferably leads to high strength, and thus the same value is desired for n₂₁ to n₂₄. When the values of n₂₁ to n₂₄ are too high, the strength of the hydrogels becomes weak, and when the values of n₂₁ to n₂₄ are too low, the hydrogels are hard to be formed owing to steric hindrance of the compound. Therefore integer values of n₂₁ to n₂₄ may be 5 to 300, preferably 20 to 250, more preferably 30 to 180, much more preferably 45 to 115, and far more preferably 45 to 55. Molecular weight of the second four-branching compound of the present invention may be 5×10³ to 5×10⁴ Da, preferably 7.5×10³ to 3×10⁴ Da, and more preferably 1×10⁴ to 2×10⁴ Da.

In the said chemical formula (II), each of R²¹ to R²⁴ is linker moiety that connects functional group and core moiety of the second four-branching compound. Each of R²¹ to R²⁴ may be the same or different, but it is preferable that each of R²¹ to R²⁴ is the same to fabricate the high-strength hydrogels with homogeneous conformation. In the chemical formula (II), R²¹ to R²⁴ are, each may be the same or different, C₁-C₇ alkylene group, C₂-C₇ alkenylene group, —NH—R²⁵—, —CO—R²⁵—, —R²⁶—O—R²⁷—, —R²⁶—NH—R²⁷—, —R²⁶—CO₂—R²⁷—, —R²⁶—CO₂—NH—R¹⁷—, —R²⁶—CO—R²⁷—, or —R²⁶—CO—NH—R²⁷, wherein R²⁵ is C₁-C₇ alkylene group, R²⁶ is C₁-C₃ alkylene group, R²⁷ is C₁-C₅ alkylene group.

In addition, when a bond between the linker moiety and the core moiety of the second four-branching compound becomes an ester linkage, the second four-branching compound is easy to be decomposed in vivo. In contrast, when the bond between the linker moiety and the core moiety of the second four-branching compound becomes an ether bond, the second four-branching compound is hard to be decomposed in vivo. In other words, decomposition properties of the second four-branching compound depend on types of R₂₁ to R₂₄. Therefore, decomposition rate of the hydrogels fabricated can be also controlled by using such a second four-branching compound. The R²¹ to R²⁴ including an ether linkage may be preferably C₁-C₇ alkylene group, preferably C₂-C₆ alkylene group, and more preferably C₃-C₅ alkylene group. The R²¹ to R²⁴ including an ester linkage is —CO—R²⁵, wherein R²⁵ indicates the C₁-C₇ alkylene group, or —CO—NH—R²⁵—, and is more preferably —CO—R²⁵, wherein R²⁵ indicates the C₃-C₅ alkylene group.

In addition, as shown in the above chemical formula (II), the desired functional group of the second four-branching compound of the present invention is N-hydroxy-succinimidyl (NHS) group. However, as mentioned above, the hydrogels of the present invention have high-strength conformation by bonding the functional group of the first four-branching compound with nucleophilicity and the functional group of the second four-branching compound with electrophilicity by chemical reaction. Therefore the other active ester groups with the electrophilicity may be used as a functional group of the second four-branching compound of the present invention. Such active ester groups include a sulfosuccinimidyl group, a Maleimidyl group, a phthalimidyl group, an imidazoyl group or a nitrophenyl group and well-known activity ester groups can be used appropriately by person skilled in the art. Each of the functional groups of the second four-branching compound may be the same or different, but the same is preferable. By making the functional groups of the second four-branching compound the same, the reactivity with the functional groups of the first four-branching compound becomes homogeneous and as a result the high-strength hydrogels with homogeneous conformation can be easily obtained.

The concentration of the second four-branching compound included in the second solution of the present invention may be 10 mg/mL to 500 mg/mL. When the concentration of the four-branching compound is too low, the strength of the gels becomes weak, and when the concentration of the four-branching compound is too high, the structure of the hydrogel becomes inhomogeneous and as a result the strength of the gels becomes weak. Therefore, 20 to 400 mg/mL are preferable, and 50 mg/mL to 300 mg/mL are more preferable, and 100 to 200 mg/mL are further more preferable.

In the method for fabricating the hydrogels in the present invention, the first and the second four-branching compounds can be mixed with mole ratio of 0.5:1 to 1.5:1. The first four-branching compound of the present invention has nucleophilic functional groups (e.g., an amino group). On the other hand, the second four-branching compound of the present invention has electrophilic functional groups (e.g., an N-hydroxy-succinimidyl (NHS) group). The functional groups of the first and second four-branching compounds of the present invention can react with each other, in which molar ratio of the reaction is 1:1. Therefore, it is more preferable that the mixed mole ratio of the first and the second four-branching compounds is nearer to 1:1. As shown in the following embodiment, 0.8:1 to 1.2:1 are desirable for the mixed mole ratio of the first and second four-branching compounds of the present invention, and 0.9-1:1.1-1 is more preferable. As shown in the following embodiment, in the fabrication method of the present invention, if the mixed mole ratio of the first and second four-branching compounds is 0.8:1 to 1.2:1, gels with higher strength than strength of cartilage (10 MPa) can be fabricated.

In the present invention, in the method for fabricating the hydrogels to control decomposition rate, two or more types of the four-branching compounds are used. As mentioned above, in the present invention, the high-strength hydrogels can be fabricated by bonding, at a mixing mole ratio of 0.8 to 1.2, the four-branching compound with an electrophilic functional group at each end and the four-branching compound with a nucleophilic functional group at each end. In addition, as mentioned above, when the bond between the core moiety of the four-branching compound and the linker moiety of the four-branching compound is the ester linkage, decomposition of the four-branching compound proceeds. In addition, when the bond between the core moiety of the four-branching compound and the linker moiety of the four-branching compound is the ether linkage, the four-branching compound remains at stable state without being decomposed. Therefore, by mixing, at a mixing mole ratio of 0.8 to 1.2, the four-branching compound with an electrophilic functional group at each end and the four-branching compound with a nucleophilic functional group at each end, the four-branching compound with the nucleophilic functional groups or the four-branching compound with the electrophilic functional groups can include ester linkage or ether linkage, respectively. In this case, the four-branching compound with the nucleophilic functional groups or the four-branching compound with the electrophilic functional groups may be two or more types of the four-branching compounds, respectively. Person skilled in art can appropriately adjust proportion including the ester linkage or the ether linkage, or which bond is used for either/both of the four-branching compound with the nucleophilic functional groups and/or the four-branching compound with the electrophilic functional groups.

In a preferable aspect of the present invention, buffer is included in the first solution or the second solution and pH of the respective solutions is adjusted. The buffer in the present invention describes liquid with capacity (pH buffer capacity) that prevents the pH in solution from changing largely. For example, the buffer of the present invention includes phosphate buffer, citric acid buffer, citric acid/phosphate buffer, acetate buffer, boric acid buffer, tartaric acid buffer, Tris buffer solution, Tris-hydrochloric acid buffer, phosphate buffered saline, or citric acid/phosphate buffered saline. In the fabrication method of the present invention, the first and the second buffer solutions may be the same or different. In addition, each of the first and the second buffer solutions may be used by mixing two or more than two types of buffer solutions. The concentration of the buffer of the present invention includes 10 mM to 500 mM. As shown in the following embodiment, in the case that buffer concentration is low, pH buffer capacity of the buffer solution is low, and control of the pH is not appropriately accomplished. On the other hand, in the case that the buffer concentration is too high, buffer component prevents formation of the hydrogels. Therefore, the concentration of the buffer of the present invention is preferably 20 to 200 mM, and more preferably 20 mM to 100 mM. When the pH of the buffer of the present invention is too strong in acidity or alkalinity, the hydrogels with homogeneous structure are not formed. Therefore it is preferable that the pH of the buffer of the present invention is 5 to 9.

The first and the second four-branching compounds of the present invention are mixed in mixing process. The mixing process of the present invention includes a step that the first solution is added to the second solution and then mixed, a step that the second solution is added to the first solution and then mixed, and a step that the first and the second solutions are mixed in equal amounts. In the fabrication method of the present invention, the addition rate and the mixing rate of the first or the second solutions are not particularly limited and can be appropriately adjusted by person skilled in art.

The mixing process of the present invention can be carried out by using a syringe for mixing two solutions, such as the one disclosed, for example, in international publication pamphlet WO2007/083522. The temperature of the two solutions at the time of the mixing is not particularly limited, and may be the temperature that each of the first and the second four-branching compounds is dissolved and these solutions are in a state where each solution is fluid. When temperature is too low, the compounds are hard to be dissolved or the fluidity of the solution is decreased, and as a result the first and the second four-branching compounds are hard to mix uniformly. On the other hand, when the temperature is too high, reactivity of the first and second four-branching compound is hard to be controlled. Therefore, in the fabrication process of the present invention, the temperature of the solution when the first and second four-branching compounds are mixed includes 1° C. to 100° C., preferably 5° C. to 50° C., and more preferably 10° C. to 30° C. In the mixing process of the present invention, each temperature of the two solutions may be different, but it is preferable that each temperature is the same because the two solutions are easy to be mixed at the same temperature.

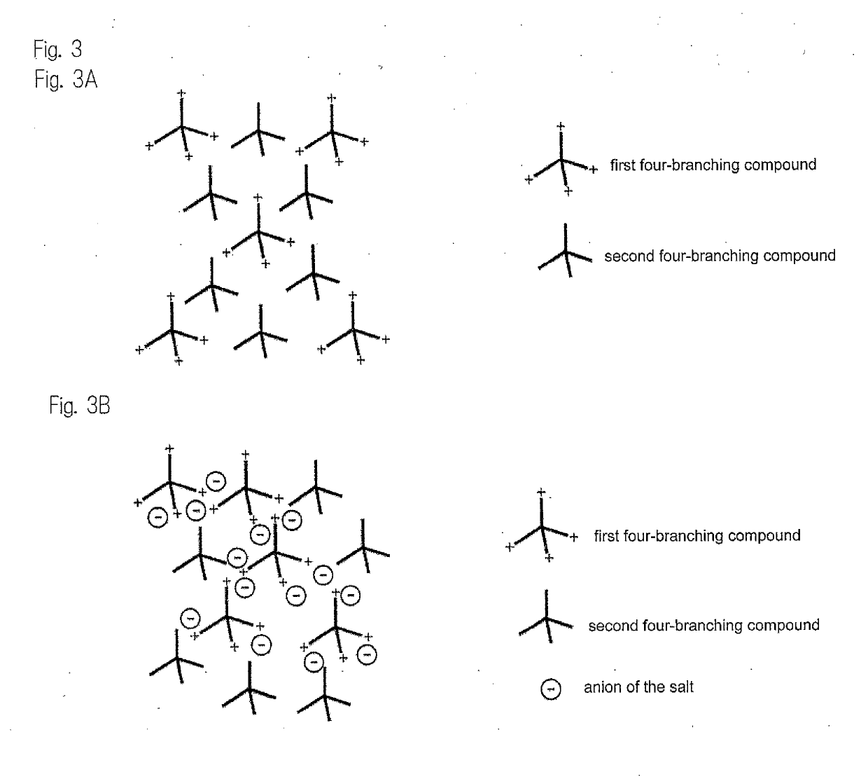

In the fabrication process of the present invention, salt concentration in the mixed solution provided by the mixing process is preferably 0 to 1×10² mM, and, more preferably 1×10⁻¹ to 1×10². As shown in the following embodiment, as the salt concentration in the mixed solution rises, ionic strength of the mixed solution rises. When the ionic strength rises, the four-branching compound does not mix homogeneously because electrostatic repulsion between positively charged amino groups is inhibited (FIG. 3B). Therefore it is preferable that the salt concentration in the mixed solution is not high. Therefore, it is preferable that the salt concentration in the mixed solution is less than or equal to 100 mM, and it is more preferable that the salt concentration in the mixed solution is less than or equal to 50 mM.

In addition, in the fabrication process of the present invention, it is preferable that the second four-branching compound stably exists without being hydrolyzed. For this reason, it is preferable that pH of the solution including the second four-branching compound is 5 to 6.5 before mixing. In addition, in the solution after mixing, to prevent inhomogeneous mixing it is preferable that 95 to 99% of the first four-branching compound exist at a state of non-cationic amino group that has ability of binding with the second four-branching compound. To undergo such a fabrication process, it is preferable that the pH of the solution just after mixing is 6 to 8. Therefore, in the fabrication process of the present invention, it is preferable that the pH of the first solution is higher than that of the second solution. The pH of the solutions can be measured by well-known method, for example, by using commercial pH meter. In this way, homogeneous and strong hydrogels can be fabricated by keeping pH at 6 to 8 after mixing and by keeping proportion of the non-cationic amino group, which can react with NHS, to 5% or less. It is noted that mixing start in the present Description is time when the first and the second solutions contact with each other.

In this way, method to raise the pH after mixing includes method to mix the first solution including the first buffer with pH of more than or equal to 7.5 and the second solution including the second buffer with pH of less than or equal to 6.5. Since the first and the second solutions of the present invention include buffer, the pH does not suddenly change by solution with different pH value. In each pH of the first and second solutions, person skilled in art can change pH after mixing by appropriately adjusting the type and the concentration of the first buffer and the second buffer included in the first and second solutions.

The second aspect of the present invention relates to the hydrogels fabricated by the method mentioned above. The hydrogels fabricated by the fabrication process of the present invention as mentioned above is high strength, and the time of gelation can be adjusted by adjusting the pH of the solution. In this way, the hydrogels of the present invention are easy to form shape fitting in introduction part, because the time to gelation can be adjusted. Therefore, as mentioned later, the hydrogels of the present invention can be suitably used as defect-filling material of bones, cartilage or intervertebral disk, and filling material for denatured parts of the bones, the cartilage, or the intervertebral disk, in orthopedic surgery of weight-bearing bones, cartilage, or intervertebral disk, such as knee cartilage operation and intervertebral disk operation. In the orthopedic surgery, the hydrogels of the present invention may be directly administered to the affected area, using a syringe for mixing two solutions mentioned above. Alternatively, the hydrogels may be formed to fit in the shape of the introduction part beforehand and then the formed hydrogels may be introduced into the affected part.

The third aspect of the present invention relates to hydrogels comprising the first and second four-branching compounds with the composition ratio of 0.5:1.0 to 1.5:1. As stated above, the nucleophilic functional group of the first four-branching compound and the electrophilic functional group of the second four-branching compound can react with each other at molar ratio of 1:1. Therefore, it is preferable that the composition ratio of the first and second four-branching compounds is near to 1:1. As shown in the following embodiment, it is preferable that the composition ratio of the first and second four-branching compounds of the hydrogels of the present invention is 0.8:1 to 1.2:1 and it is more preferable that the composition ratio is 0.9-1:1.1-1. As shown in the following embodiment, in the fabrication method of the present invention, if mixing mole ratio of the first and second four-branching compounds is 0.8:1 to 1.2:1, the gels whose strength is more than that of cartilage (10 MPa) can be fabricated. As for the hydrogels fabricated by such a fabrication method, neutron scattering curve of the hydrogels can be fitted by the Ornstein-Zernike (OZ) function. In this way, it can be evaluated whether the structure of the hydrogels is homogeneous.

“The neutron scattering curve of the hydrogels can be fitted by the Ornstein-Zernike (OZ) function” means that approximation curve obtained from the group of values measured by the neutron scattering for the hydrogels correlates to not “combination curve between theoretical curves expressed with Gauss function and the OZ function” but “the theoretical curve expressed with the OZ function”. That the approximation curve obtained from the group of the values measured by the neutron scattering for the hydrogels correlates to theoretical curve expressed with the OZ function can be evaluated by curve fitting. Specifically, when the theoretical curve expressed with the OZ function is overlapped with the approximation curve obtained from the group of the values measured by the neutron scattering so that the overlap is largest, degree of the overlap (degree of the fitting) is preferably more than or equal to 80% and more preferably more than or equal to 90%. In this way, method to evaluate the degree of the fitting by overlapping the two curves is well-known and it can be appropriately performed by person skilled in art.

The third favorable aspect of the present invention is hydrogels that compression breaking strength is more than or equal to 10 MPa. The compression breaking strength of the hydrogels of the present invention can be examined by well-known method, using well-known measuring equipment. An equipment for measuring the compression breaking strength includes, for example, compression tester (Instron 3365) made in Instron company. The compression breaking strength is maximum stress that a gel sample breaks when compressive load was applied to the gel sample. The compression breaking strength can be expressed with the value of compressive force, which is of when uniaxial loading is applied to a columnar gel sample, divided by cross section that is perpendicular to the axis. It is preferable that the strength of the hydrogels of the present invention is more than the compression breaking strength 10 MPa of the cartilage in a living body. The hydrogels with such a compression breaking strength can be used in defective and denatured parts of bones which are subject to weight-bearing.

Because the hydrogels of the present invention are high strength and time to gelation can be adjusted, these can be suitably used in defective part of bones, cartilage or intervertebral disk or denatured part of the bones, the cartilage or the intervertebral disk, such as knee cartilage or the intervertebral disk, which are subject to weight-bearing in a living body. In addition, the gels of the present invention can adjust time to gelation by adjusting the pH of the solution. In addition, as disclosed in international publication pamphlet WO2007/083522, on-site gel infusion is enabled if a syringe for mixing two liquids is used. Therefore, the hydrogels of the present invention can provide a new regimen in orthopedic surgery and so on. In the current operation to reinforce the knee cartilage and the intervertebral disk, skin is cut open, the affected part is opened, and then the gels are introduced into the opened part. In contrast, as for the hydrogels of the present invention, the dosage of the gels is enabled by using method of discography. The method of the discography is a method that the gels are poured from posterior direction, using a needle for inserting into the intervertebral disk. In this way, because the gels can be poured into nucleus pulposus of the intervertebral disk without skin incision, low invasive surgery that burden to patient's body is low can be carried out. In this way, the hydrogels of the present invention have mechanical property of the intervertebral disk for the short term, and are useful new material that is expected to have protective efficacy for intervertebral degeneration for the long term.

In addition, in the hydrogels of the present invention, the gels may be poured on-site after diskectomy (LOVE method) or the operation for endoscopic extraction of nucleus pulposus. The hydrogels of the present invention are injected on-site and time to gelation can be adjusted. Therefore, the gelation can be artificially adjusted to gelate in a state of fitting in shape of the affected part. Therefore, postoperative early recovery can be expected and postoperative degeneration in the intervertebral disk can be also prevented.

Furthermore, the hydrogels of the present invention can be used as a model of hernia. As for the hernia model, with approach to front and lateral side of lumbar vertebrae, front surface of body of vertebra is extended by entering from rear of retroperitoneal, and nucleus pulposus is aspirated by using 18 G (gage) or 20 G (gage) needle and a 10 mL syringe, and then the gels are injected, and the progress can be observed.

In other words, the present invention provides not only therapeutic treatment of defective parts of bones, cartilage, or intervertebral disk by using the hydrogels, wherein the composition ratio of the first four-branching compound and the second four-branching compound is 0.8:1 to 1.2:1, but also the therapeutic treatment of degenerated parts of the bones, the cartilage, or the intervertebral disk by using the hydrogels, wherein the composition ratio of the first four-branching compound and the second four-branching compound is 0.8:1 to 1.2:1. The hydrogels of the present invention have mechanical property of intervertebral disk for the short term, and protective efficacy for intervertebral degeneration is expected for the long term.

### Embodiment 1

Fabrication of Four-Branching Compound.

Two four-branching compounds, TAPEG (tetraamine-polyethylene glycol) and TNPEG (N-hydroxy-succinimidyl-polyethylene glycol (NHS-PEG)) were obtained by aminating and succin-imidizing THPEG (tetrahydroxyl-polyethylene glycol) which has hydroxyl groups at each end.

Fabrication of THPEG

Pentaerythritol (0.4572 mmol, 62.3 mg) as an initiator was dissolved in mixed solvent of DMSO/THF (v/v=3:2) of 50mL, and potassium naphlene (0.4157 mmol, 1.24 mg) as an metalating agent was used, and ethylene oxide (200 mmol, 10.0 mL) was added, followed by being heated and stirred at 60° C. under Ar atmosphere for approximately two days. After the reaction is completed, precipitate was provided by reprecipitating with diethyl ether and then by filtration. Furthermore, it was washed with diethyl ether three times, and the obtained white solid was dried under reduced pressure, and then the THPEG of 20 k was obtained.

Fabrication of TAPEG

After the THPEG (0.1935 mmol, 3.87 g, 1.0 equiv) was dissolved in benzene and then freeze-dried, it was dissolved in THF of 62 mL and triethylamine (TEA) (0.1935 mmol, 3.87 g, 1.0 equiv) was added to it. THF of 31 mL and methanesulphonyl chloride (MsCl) (0.1935 mmol, 3.87 g, 1.0 equiv) were added to a different recovery flask (egg plant flask) and it was immersed in ice-bath. After THF solution of MsCl was dripped to the THF solution of the THPEG and TEA for approximately one minute, and it was stirred in ice bath for 30 minutes, and then was stirred at room temperature for one and a half hours. After the reaction was completed, precipitate was provided by reprecipitating with diethyl ether and then by filtration. Furthermore, it was washed with diethyl ether three times, and the obtained white solid was moved to a recovery flask (egg plant flask), and 25% ammonium hydroxide of 250 mL was added to it and it was stirred for four days. After the reaction was completed, solvent was distilled under reduced pressure by evaporator, and then it was dialyzed by using water as external solution two or three times, and then freeze-dried, and then the white solid of the TAPEG was obtained. The chemical formula of the fabricated TAPEG is shown in the chemical formula (Ia). In the chemical formula (Ia), n₁₁ to n₁₄ were an integer that is any one of 50 to 60 if molecular weight of the TAPEG is approximately 10,000 (10 kDa) and 100 to 115 if the molecular weight is approximately 20,000 (20 kDa).

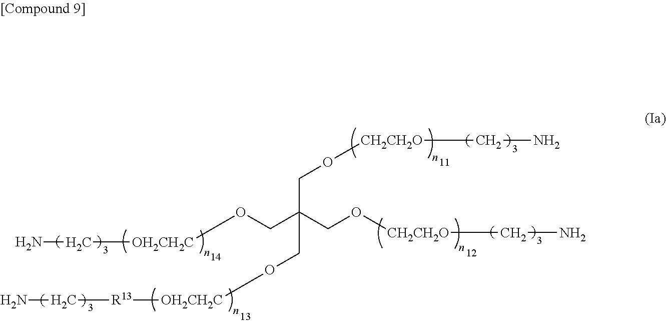

Fabrication of TNPEG

THPEG (0.2395 mmol, 4.79 g, 1.0 equiv) was dissolved in THF, 0.7 mol/l glutaric acid/THF solution (4.790 mmol, 6.85 mL, 20 equiv) was added to it, and then it was stirred for six hours under Ar atmosphere. After the reaction was completed, it was dripped to 2-propanol and was subjected to centrifuge three times. The obtained white solid was moved to the 300 mL recovery flask (egg plant flask), and solvent was distilled under reduced pressure by evaporator. The residue was dissolved in benzene and impurities were removed by filtration. By removing solvent by freeze-drying the obtained filtrate, a white solid of Tetra-PEG-COOH whose end is modified by carboxyl group was obtained. This Tetra-PEG-COOH (0.2165 mmol, 4.33 g, 1.0 equiv) was dissolved in THF, N-hydrosuccinimide (2.589 mmol, 0.299 g, 12 equiv), and N,N′-diisopropyl succinimide (1.732 mmol, 0.269 mL, 8.0 equiv) were added to it, and then it was heated and stirred at 40° C. for three hours. After the reaction was completed, solvent was distilled under reduced pressure by evaporator. It was dissolved in chloroform and the extraction was made three times with saturated salt water, and chloroform layer was extracted. Furthermore, it was dehydrated with magnesium sulfate, and was filtered, and then solvent was distilled under reduced pressure by evaporator. The obtained residue was freeze-dried with benzene, and then the white solid of the TNPEG was obtained. The chemical formula of the fabricated TNPEG is shown in the chemical formula (IIa). In the chemical formula (IIa), n₂₁ to n₂₄ were an integer that is any one of 45 to 55 if molecular weight of the TNPEG is approximately 10,000 (10 k) and 90 to 115 if the molecular weight of the TNPEG is approximately 20,000 (20 k).

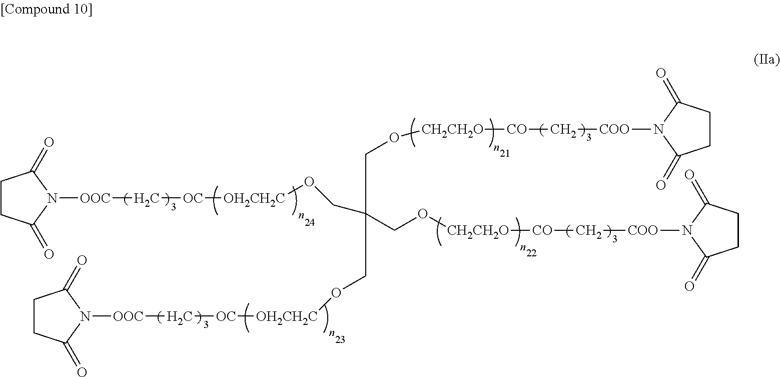

### Embodiment 2

Effect of Solvent on Strength of the Gels.

Each of TAPEG (Ia) (10 k) and TNPEG (IIa) (10 k) was dissolved in pure water, phosphate buffer (pH 7.4), phosphate buffered saline (PBS), and saline, at concentration of 100 mg/mL. After the preparation, the two obtained solutions were immediately mixed, and it was then gelated at 37° C., and after the gelation gel strength was measured. A penetrating rod of 2 mm in diameter was penetrated into a cylindrical sample of 15 mm in diameter and 7.5 mm in height, and pressure in penetration of 98% was used as strength.

As a result, all the gels were not broken even at deformation of 100%, and thus the gels can be said to be unbreakable even at large deformation. As for gelation rate, the gelation in pure water was the fastest and it took only tens of seconds. The gelation in phosphate buffer the second fastest and the gelation in PBS was the third fastest, and the gelation in saline was the slowest and it took around five minutes. The result of the gel strength was shown in Table 1.

TABLE 1

| | Gel strength | |
|---|---|---|
| Solvent | (kPa) | Break or non break |
| 20 mM phosphate buffer | 16.6 | Non break |
| (pH 7.4) | | |
| PBA of pH 7.4 | 12.2 | Non break |
| Pure water | 6.7 | Non break |
| saline | 4.5 | Non break |

In the present invention, reaction rate is very important. On one hand, if the reaction is too fast, the viscosity of the solution becomes high before the four-branching compounds are mixed homogeneously, and as a result homogeneous network structure cannot be obtained. On the other hand, if the reaction is too slow, degradable active ester linkages are hydrolyzed and as a result reaction yield is low. Therefore, because the gels fabricated in pure water are formed before mixing, network structure becomes inhomogeneous and it is thought that strength of the gels is weak. ***In other words, because the gels fabricated in pure water are formed before mixing, network structure becomes inhomogeneous and it is thought that strength of the gels is weak.*** On the other hand, because in the gels fabricated in saline active ester linkages were hydrolyzed during the reaction, the reaction yield decreases and it is thought that strength of the gels decreases. Therefore, in the phosphate buffer and the PBS, both of which lead to an intermediate reaction rate, the reaction yield is high and it is thought that mechanical strength rose.

### Embodiment 3

Effect of Solvent pH on Gelation Strength and Gelation Time.

Each of TAPEG (Ia) (10 k) and TNPEG (IIa) (10 k) was dissolved in phosphate buffer (pH 6.0,7.4,9.0) and citric acid buffer (pH 6.0,7.4,9.0), at concentration of 100 mg/mL. After the preparation, the two obtained solutions were immediately mixed, and it was then gelated at 37° C., and after the gelation gel strength was measured. A penetrating rod of 2 mm in diameter was penetrated into a cylindrical sample of 15 mm in diameter and 7.5 mm in height, and pressure in penetration of 98% was used as strength. As a result, all the gels were not broken even at deformation of 100%. Higher the pH is, faster the gelation rate is, and the gelation was completed within one minute at pH 9.0 and for around five minutes at pH 6.0. The result was shown in Table 2.

TABLE 2

| | Gel strength | |
|---|---|---|
| Solvent | (kPa) | Break or non break |
| Phosphate buffer (pH 6.0) | 9.52 | Non break |
| Phosphate buffer (pH 7.4) | 19.3 | Non break |
| Phosphate buffer (pH 9.0) | 14.2 | Non break |
| Citric acid buffer (pH 6.0) | 8.6 | Non break |
| Citric acid buffer (pH 7.4) | 12.9 | Non break |
| Citric acid buffer (pH 9.0) | 10.2 | Non break |

As a result, when solvent whose pH leads to an intermediate reaction rate was used, the hydrogels with high strength was obtained. Around pH 7.4 is thought to be an optimum value. In addition, because citric acid buffer solution has lower buffer capacity at around pH 7 than phosphate buffer solution, pH control was not worked and as a result such a result is thought to be obtained. Therefore, phosphate buffer having high buffer capacity at around pH 7 is thought to be most suitable.

### Embodiment 4

Effect of Buffer Concentration on Gel Strength and Gelation Time.

Each of TAPEG (10 k), TNPEG (10 k) was dissolved in phosphate buffer (pH7.4, 2 mM, 20 mM, 100 mM, 200 mM) and citric acid buffer (pH7.4, 2 mM, 20 mM, 100 mM, 200 mM), at the concentration of 100 mg/mL. After the preparation, the two obtained solutions were immediately mixed, and it was then gelated at 37° C., and after the gelation gel strength was measured. A penetrating rod of 2 mm in diameter was penetrated into a cylindrical sample of 15 mm in diameter and 7.5 mm in height, and pressure in penetration of 98% was used as strength.

As a result, all the gels were not broken even at deformation of 100%. Higher buffer concentration was, faster the gelation was, but all the gels were gelated for about 1 or 2 minutes. The result was shown in Table 3.

TABLE 3

| | Gel strength | |
|---|---|---|
| Solvent | (kPa) | Break or non break |
| Phosphate buffer (pH 7.4 2 mM) | 6.7 | Non break |
| Phosphate buffer (pH 7.4 20 mM) | 19.3 | Non break |
| Phosphate buffer (pH 7.4 100 mM) | 15.7 | Non break |
| Phosphate buffer (pH 7.4 200 mM) | 13.0 | Non break |
| Citric acid buffer (pH 7.4 2 mM) | 6.3 | Non break |
| Citric acid buffer (pH 7.4 20 mM) | 12.9 | Non break |
| Citric acid buffer (pH 7.4 100 mM) | 8.8 | Non break |
| Citric acid buffer (pH 7.4 200 mM) | 8.5 | Non break |

As a result, because the reaction rate did not change significantly, the buffer concentration is thought not to influence significantly the reaction rate. However, the gel strength was high at buffer concentration from 20 mM to around 100 mM. In case that buffer concentration is low, buffering limit of the buffer solution was too low to control the pH, and then the gelation becomes faster and it is thought that thereby homogeneous structure was not obtained. In other words, in the case that the four-branching compound has the concentration of 100 mg/mL, if the concentration of the buffer is more than 20 mM, the solution can be kept at appropriate pH. In contrast, the reason why strength decreased in highly-concentrated region is thought to be because the four-branching compounds were not mixed homogeneously. At around pH 7, because the amino groups are protonated and they have positive charge, the amino groups are repelled from each other. Mixing of the TAPEG (Ia) and the TNPEG (IIa) is thought to be promoted by this repulsion. Because ionic strength is high in the case that buffer concentration is high, the repulsion between amino groups is inhibited and then the mixture did not become homogeneous, and thus it is thought that inhomogeneous structure was obtained.

### Embodiment 5

Effect of Salt Concentration on Gel Strength and Gelation Time.

Each of TAPEG (Ia) (10 k) and TNPEG (IIa) (10 k) was dissolved in aqueous solutions, in which sodium chloride was dissolved to concentrations of 0 mM, 50 mM, 100 mM, and 200 mM, and phosphate buffer (pH7.4, 20 mM), at concentration of 100 mg/mL. After the preparation, the two obtained solutions were immediately mixed, and it was then gelated at 37° C., and after the gelation gel strength was measured. A penetrating rod of 2 mm in diameter was penetrated into a cylindrical sample of 15 mm in diameter and 7.5 mm in height, and pressure in penetration of 98% was used as strength.

As a result, all the gels were not broken even at deformation of 100%. Higher ionic strength was, slower the gelation rate was. In addition, the reaction rate was fast in pure water and thus the gelation was completed within one minute. In contrast, the gelation in the phosphate buffer was completed for around 1 or 2 minutes. The result was shown in Table 4.

TABLE 4

| | NaCl concentration | Gel strength |
|---|---|---|
| Solvent | (mM) | (kPa) |
| Phosphate buffer (pH 7.4 20 mM) | 0 | 19.3 |
| Phosphate buffer (pH 7.4 20 mM) | 50 | 13.4 |
| Phosphate buffer (pH 7.4 20 mM) | 100 | 13.8 |
| Phosphate buffer (pH 7.4 20 mM) | 200 | 11.2 |
| Pure water | 0 | 6.2 |
| Pure water | 50 | 7.1 |
| Pure water | 100 | 5.6 |
| Pure water | 200 | 5.7 |

In both cases of using pure water and of using phosphate buffer, when salt concentration was high, the strength of the gels decreased. It is thought that this is because electrostatic repulsion between amino groups was inhibited by a rise in ionic strength and thereby the mixed state of the four-branching compounds became inhomogeneous.

### Embodiment 6

Optimization Experiment of Solvent for Gel Fabrication.

Both TAPGE (Ia) (10 k) and TNPEG (IIa) (10 k) were dissolved in phosphate buffer (pH7.4, 50 mM), and only TAPEG (Ia) was dissolved in phosphate buffer (pH7.4, 50 mM), and only TNPEG (IIa) was dissolved in citric acid/ phosphate buffer (pH5.8, 5.0 mM), at concentration of 100 mg/mL. After the preparation, the two obtained solutions were immediately mixed, and it was then gelated at 37° C. The gel was formed to have a cylindrical shape of 15 mm in diameter and 7.5 mm in height and then the compressive elastic modulus of the gels was measured.

As a result, the elastic modulus of the gels fabricated by dissolving the TAPEG (Ia) in the phosphate buffer (pH7.4, 50 mM) and by dissolving the TNPEG (IIa) in the citric acid/phosphate buffer (pH5.8, 50 mM) was higher. The result was shown in Table 5.

TABLE 5

| | | Compressive elastic |
|---|---|---|
| Solvent in TAPEG | Solvent in TNPEG | modulus (kPa) |
| Phosphate buffer (pH 7.4 | Phosphate buffer (pH 7.4 | 90.3 |
| 20 mM) | 20 mM) | |
| Phosphate buffer (pH 7.4 | citric acid/phosphate | 98.7 |
| 20 mM) | buffer(pH 5.8, 20 mM) | |

In a high pH state, the active ester linkage of the TNPEG (IIa) is hydrolyzed and does not contribute to the reaction. It is thought that because the hydrolysis was able to be restrained by lowering only the pH of the TNPEG solution, the final reaction yield was improved.

### Embodiment 7

Examination of Mixing Ratio of TAPEG and TNPEG.

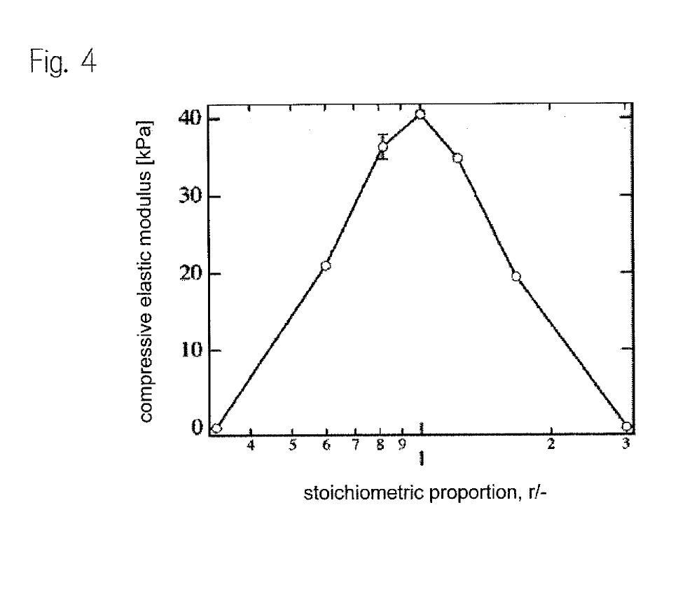

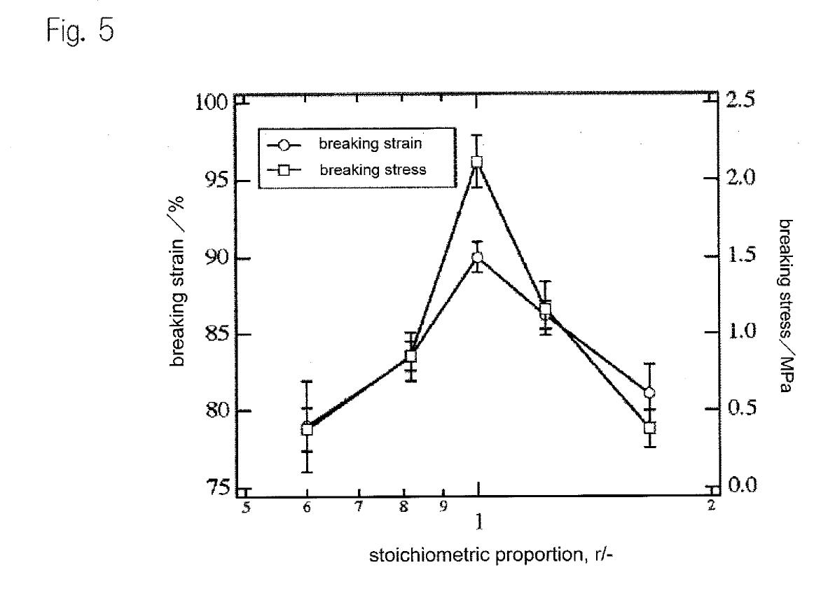

Given quantities of TAPEG (Ia) (molecular weight 10 k) and TNPEG (IIa) (molecular weight 10 k) ((total dose of the precursor)=600 mg) were respectively dissolved in 100 mM phosphate buffers (10 mL) of pH 7.2 and pH 7.4. Each solution of the compounds was mixed in equal volume at room temperature in order that mole fraction of the TAPEG (Ia) and the TNPEG (IIa) becomes 0.33 to 3.0, and the gelation was carried out for two hours, and then the gels were formed to have a cylindrical shape of 15 mm in diameter and 7.5 mm in height. Compression test was carried out at a rate of 0.75 mm/min by using a mechanical testing machine (INSTRON3365 made in Instron Corporation). The result was shown in FIGS. 4 and 5.

FIG. 4 illustrates compressive elastic modulus (kPa) of the mixed gels in which mole fraction (r) of TAPEG (Ia) and TNPEG (IIa) is in the range of 0.33 to 3.0. FIG. 5 illustrates breaking strain (%) and breaking strength (MPa) of the mixed gels in which mole fraction of the TAPEG (Ia) and the TNPEG (IIa) is in the range of 0.6 to 1.4. From results of FIGS. 4 and 5, it was indicated that maximum of the compressive elastic modulus and the breaking strength was when r=1.0 and thus the four-branching compounds reacted on an equimolar basis with each other. In addition, it was shown that when there is excess or deficiency of one component, the gels become weak. Furthermore, even if there is excess or deficiency of one component the values of the compressive elastic modulus at r and at inverse of r are almost the same to each other and the values of the compressive elastic modulus decreased in the same way. This suggests that network structures are similar to each other. Such a stoichiometry characteristics and the gelation process that is high in symmetry are unprecedented, and it is thought that homogeneous network structure of the hydrogels is formed. It was shown that the optimum amount and the optimum ratio of the four-branching compounds were required to form homogeneous network structure.

From FIGS. 4 and 5, it was shown that when mole fraction of TAPEG (Ia) and TNPEG (IIa) is in the range of 0.6 to 1.4, the gels with breaking strength of more than 0.8 MPa is obtained (FIG. 5). In addition, it was show that when the mole ratio is 0.8 to 1.2, compressive elastic modulus becomes about 40 kPa (FIG. 4), high breaking strength of about 1 MPa is achieved, and it can be favorably used as biomaterial (FIG. 5). Therefore, it was shown in the hydrogels of the present invention that by having composition ratio TAPEG (Ia) and TNPEG (IIa) in the range of 0.6:1 to 1.4:1 and preferably in the range of 0.8:1 to 1.2:1, the hydrogels with homogeneous network structure are formed.

### Embodiment 8

Measurement of Compression Breaking Strength.

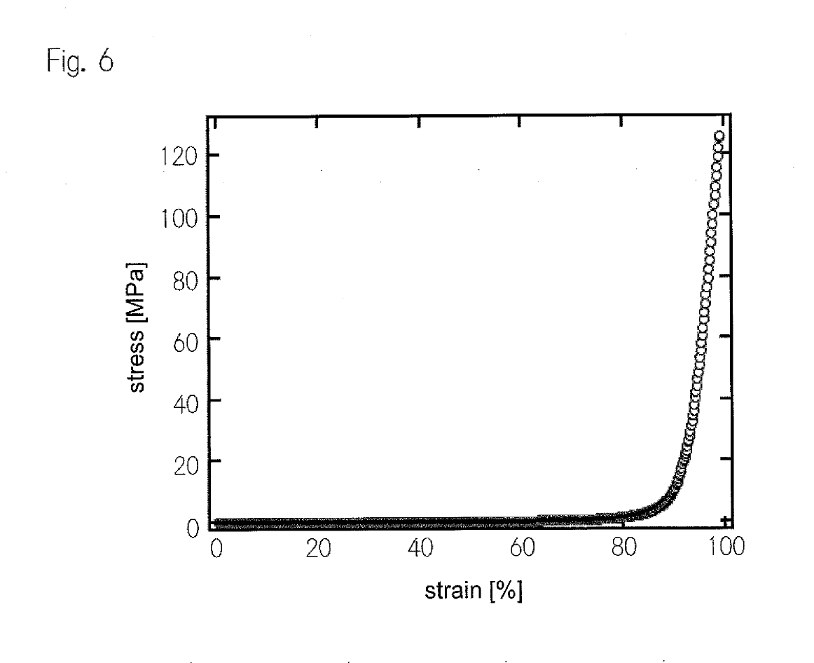

TAPEG (Ia) and TNPEG (IIa) of molecular weight 20,000 were dissolved in phosphoric acid buffer of 100 mM and citric acid/a phosphate-buffered solution, at concentration of 160 mg/mL, and then the two solutions were mixed, and clear colorless transparent hydrogels were formed in around one minute. A cylindrical sample of 7 mm in diameter and 3.5 mm in height was fabricated, and compressive strength test was carried out by using a compression tester (Instron). The result was shown in FIG. 6. The vertical axis of FIG. 6 shows stress [MPa], and the horizontal axis shows strain [%] of the hydrogels. As a result, this hydrogel was not broken even at distortion of more than 90% and was also able to withstand a stress of more than 100 MPa. This value not only exceeds the strength of the conventional hydrogels but also exceeds by far 10 MPa that is breaking stress of the cartilage in a living body, and thus it is thought that the application to not only articular cartilage but also intervertebral disk and others which is subject to weight-bearing is possible.

### Embodiment 9

Analysis of Homogeneity of Network Structure by Neutron Scattering Measurement.

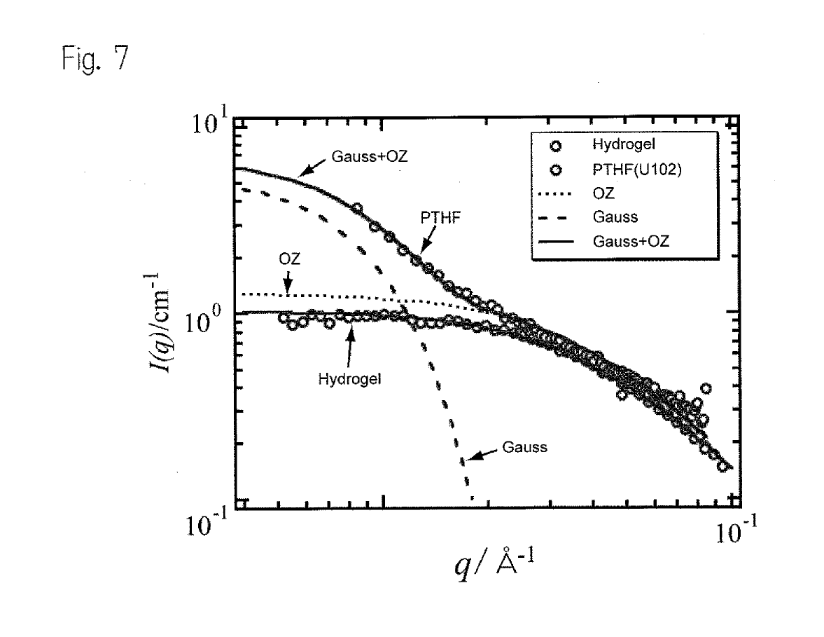

TAPEG (Ia) and TNPEG (IIa) of molecular weight 10,000 were dissolved in phosphate-buffered solution (pH 7.4) of 50 mM and citric acid/phosphate-buffered solution (pH 5.8), at various concentrations, and the hydrogels were fabricated by mixing the two solutions. For the obtained hydrogels, neutron scattering measurement was performed to analyze inhomogeneity in the structure. The result was shown in FIG. 7.

“Gauss+OZ” in FIG. 7 indicates scattering curve of the normal hydrogels (Example: PTHF (U102)), it can be described by adding Ornstein-Zernike (OZ) function based on thermal fluctuations in polymer and Gauss function representing excess scattering caused by inhomogeneity existing in the system. The “Gauss” in FIG. 7 shows Gaussian function curve that represents excess scattering of when the gels are inhomogeneous. The “OZ” in FIG. 7 indicates the OZ function curve representing the neutron scattering of when the gels are homogeneous. “The hydrogel” in FIG. 7 indicates the hydrogel of the present invention. As shown in FIG. 7, in normal hydrogels, the contribution of the Gaussian function corresponds to upturn of the curve in the small angle region. In contrast, scattering function obtained from the hydrogels of the present invention did not contribute to the Gauss function at all and description with only the OZ function was possible. Such an experimental result is not observed even in any hydrogels obtained so far, which strongly supports that the present hydrogels have unprecedented and very homogeneous structure. This remarkable homogeneity is thought to contribute significantly to high mechanical strength of the hydrogels.

### Embodiment 10

Test of Subcutaneous Implantation into Mouse Back.

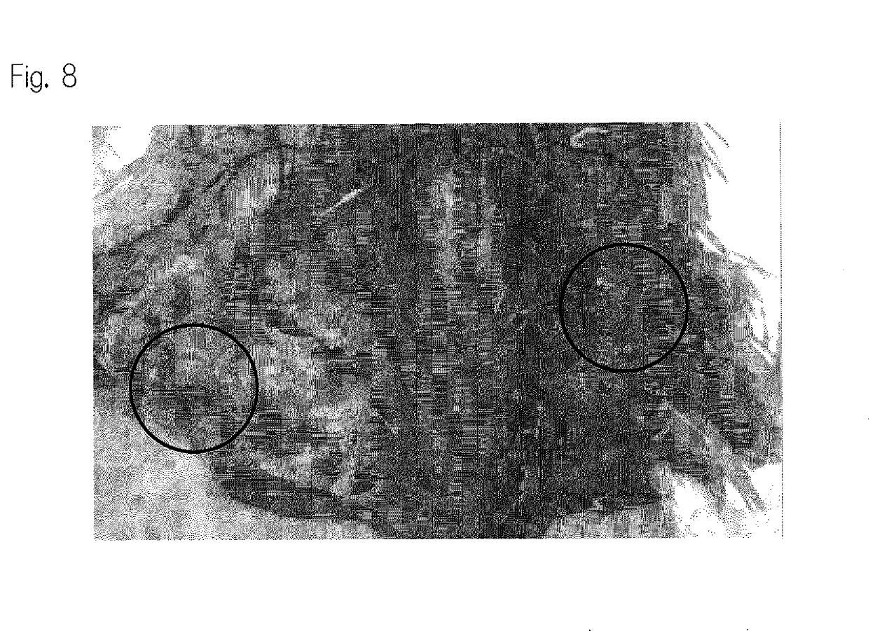

TAPEG (Ia) and TNPEG (IIa) of molecular weight 20,000 were dissolved in phosphate-buffered solution (pH 7.4) of 100 mM and citric acid/a phosphate-buffered solution (pH 5.8), at concentration of 160 mg/mL. The obtained solution was loaded to the syringe for mixing two solutions and injected to the back of C57BL/6 mouse. Then, the occurrence of gelation in mouse subcutis was confirmed by palpation. At one month after implantation, the mouse was dissected and the follow-up study was carried out for the implanted part. A photograph of the implanted part was shown in FIG. 8. As a result, neither inflammatory reaction nor toxic response were observed.

### Embodiment 11

Implantation Test into Knee Cartilage of Dog.

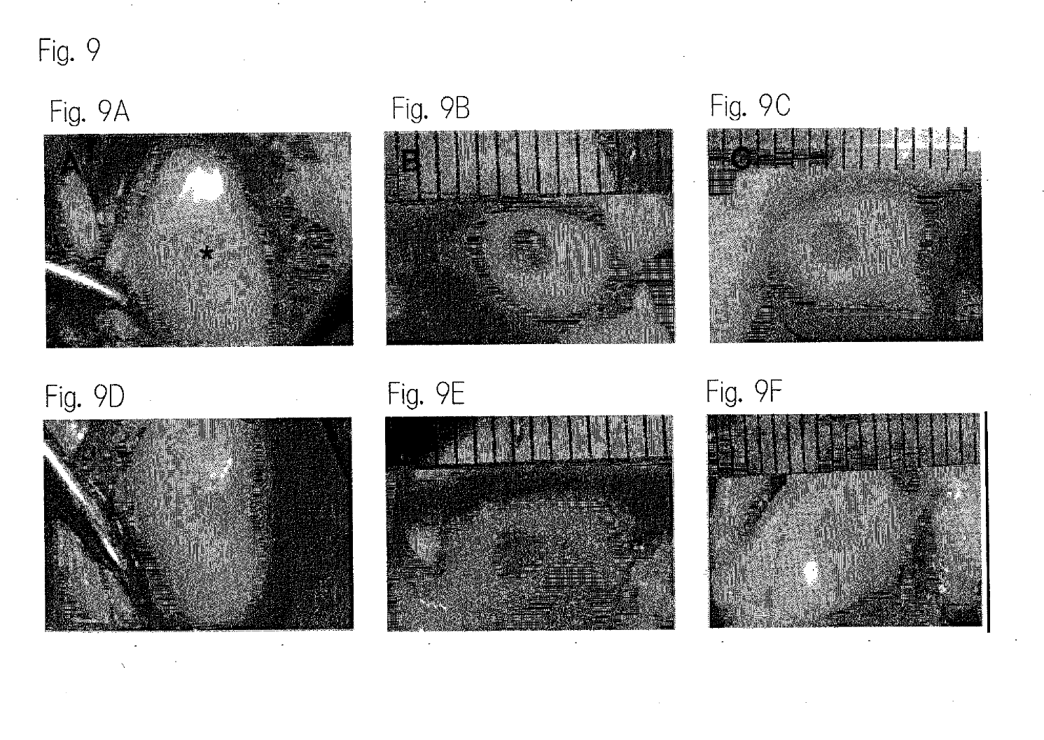

To test the application to disease in articular cartilage, a defect of 3 mm in diameter was fabricated at knee cartilage of a dog and the gels were fabricated on-site by using a syringe for mixing two solutions. At two months and four months after surgery, dissection was carried out and the implanted part was observed. The result was shown in FIG. 9. FIGS. 9A to 9C show the implanted part two months later after surgery, and FIGS. 9D to 9F show the implanted part four months later after surgery. As a result, the hydrogels remained in the affected area, and the inflammatory reaction and the toxic response were not observed.

### Embodiment 12

Implantation Test into Intervertebral Disk of Swine.

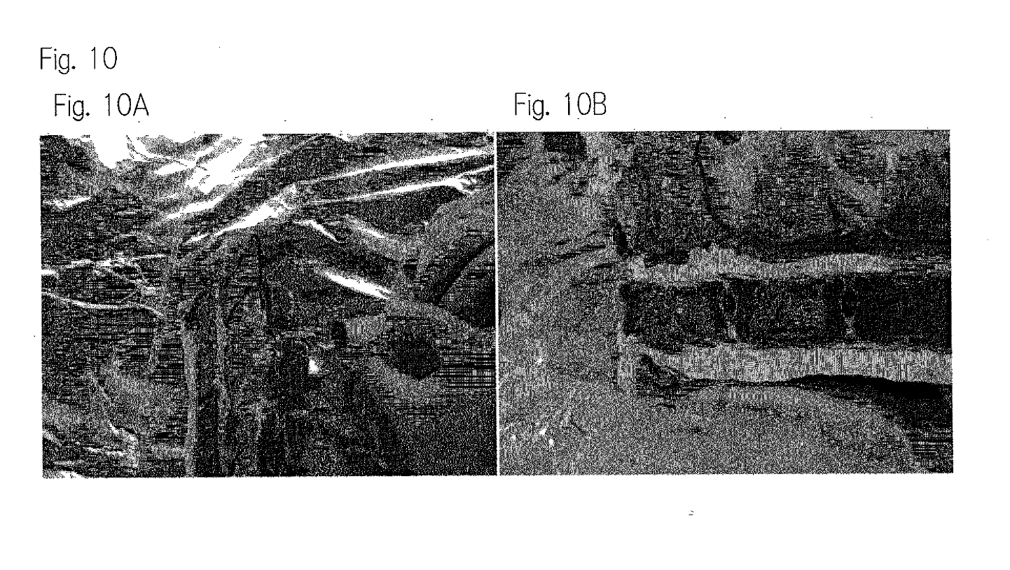

To test the application as filler into intervertebral disk, nucleus pulposus was removed from the intervertebral disk of a swine and then hydrogels were fabricated in the air gap by using a syringe for mixing two solutions. The fabrication of the hydrogels was possible in the air gap part in which the nucleus pulposus of the intervertebral disk was removed. The result was shown in FIG. 10. FIG. 10A shows a photograph of implantation in progress, and FIG. 10B shows a photograph of the intervertebral disk after the implantation.

### Embodiment 13

Examination of Decomposition Rate of Gels.

To examine decomposition rate of the gels, three types of the four-branching compounds, TAPEG (Ia) (the following chemical formula (Ia)), TNPEG (IIa) (the following chemical formula (IIa)) and TNPEG (IIb) (the following chemical formula (IIb)), were used.

In the chemical formula (Ia), n₁₁ to n₁₄ were 50 to 60, and molecular weight was approximately 10,000 (10 k).

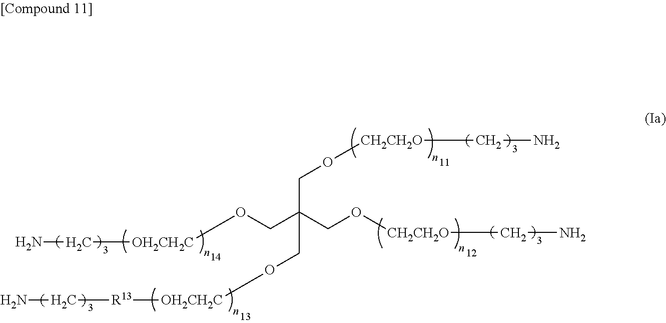

In the chemical formula (IIa), n₂₁ to n₂₄ were 45 to 55, and molecular weight was approximately 10,000 (10 k).

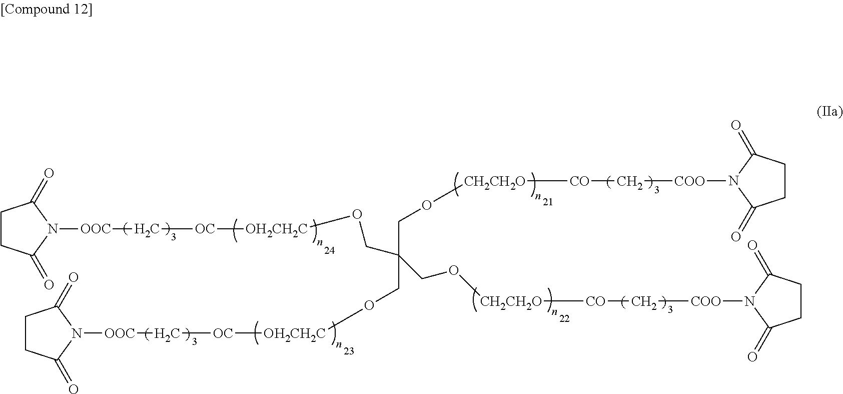

In the chemical formula (IIb), n₂₁ to n₂₄ were 45 to 55, and molecular weight was approximately 10,000 (10 k).

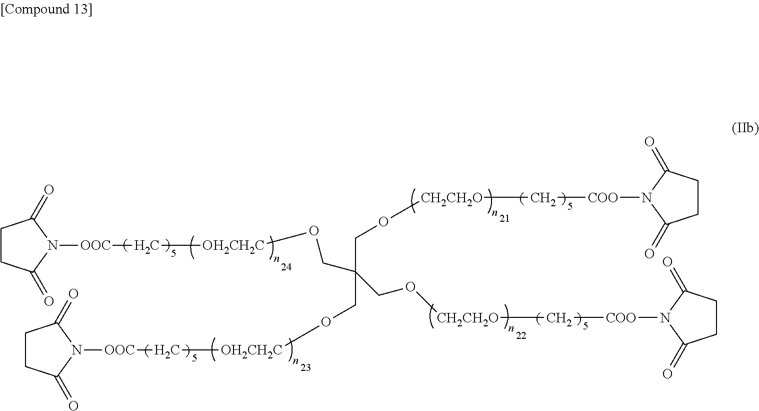

The above three types of the compounds were dissolved in phosphate buffer (pH7.4, 20 mM) to form 60 mg/mL. Each combination and mixing ration were shown in Table 6. According to the ratio of the following Table 6, each was mixed to form the gels.

TABLE 6

| | TAPEG(Ia) | TNPEG(IIa) | TNPEG(IIb) |
|---|---|---|---|
| Pattern 1 | 1 | 1 | — |
| Pattern 2 | 1 | — | 1 |
| Pattern 3 | 2 | 1 | 1 |

Comparison result of mechanical strength of three types of the gels fabricated was shown in Table 7.

TABLE 7

| | | | Compressive |
|---|---|---|---|
| | Breaking strain | Breaking strength | elastic modulus |
| Sample | (%) | (MPa) | (kPa) |
| Pattern 1 | 88.6 | 2.12 | 92.7 |
| Pattern 2 | 84.6 | 1.78 | 85.2 |
| Pattern 3 | 88.7 | 1.92 | 91.7 |

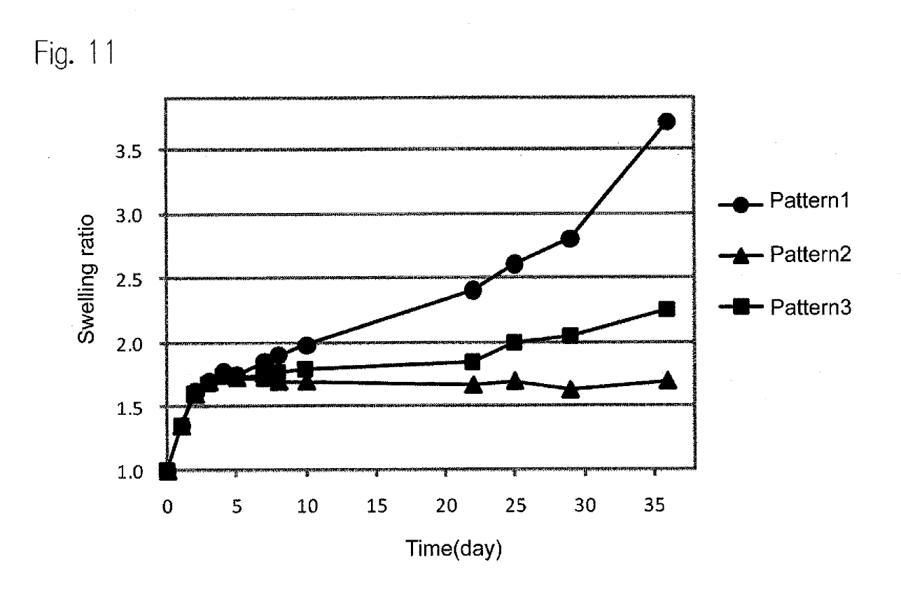

As understood from the result of Table 5, breaking strain, breaking strength, compressive elastic modulus of three types of hydrogels were almost the same to each other. Using these gels, decomposition rate of the gels was examined. Three types of the gels fabricated were left to stand at 37° C. in simulated body fluid and swelling ratio of the gels was measured. The result was shown in FIG. 11. The vertical axis shows swelling ratio, and the horizontal axis shows the number of days for which it was left to stand. It is shown that the higher the swelling ratio is, the more the gels are decomposed. As shown in FIG. 11, in the pattern 2, the swelling ratio was constant after swelling to some extent. In other words, it was shown that the gels are hardly decomposed. In the pattern 1, it was shown that the swelling ratio increased with the number of days and the gels were decomposed with the number of days, and the gels were completely decomposed two months later although this was not shown in FIG. 11. The pattern 3 showed intermediate behavior between the patterns 1 and 2. From this, it was shown that decomposition rate of the gels can be controlled by changing mixing ratio of the TNPEG (IIa) and the TNPEG (IIb).

### Embodiment 14

Examination of Cell Proliferation Activity in the Presence of Hydrogels.

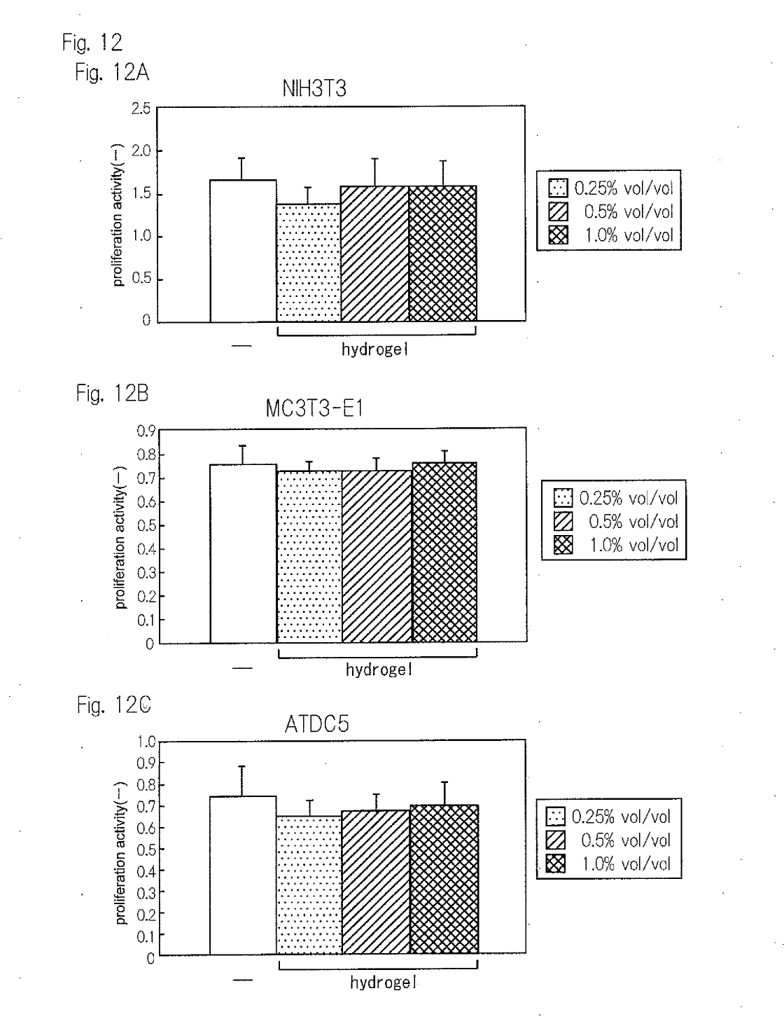

Each of fibroblast cell line of mouse, NIH3T3, precursor cell line of mouse cartilage, ATDC5, and osteoblast cell line of mouse, MC3T3-E1 was seeded on 12-well plate at cell density of 40,000 cells/2 mL/well and was cultured for 24 hours. In addition, Dulbecco's Modified Eagle Medium (DMEM) (made in Sigma company) including 10% FBS (made in Gibco company) and 1% penicillin/streptomycin was used as culture medium. After each cell was culture for 24 hours, the culture medium was changed for fresh medium. The hydrogels equivalent to 0.25% vol/vol, 0.5% vol/vol, and 1.0% vol/vol of the culture medium were immersed in the culture medium by using Transwell, and then cultured for 24 hours. In addition, the hydrogels of any one of combinations in the pattern 2 in Table 6 was used. For each cell, cell proliferation activity was measured by using Cell counting kit-8 (made in Wako company). The cell proliferation activity was examined by measuring absorbance (OD450 nm) of each well. The result was shown in FIG. 12.

FIG. 12 illustrates cell the proliferation activity (n=6) in each cell of the NIH3T3, the MC3T3-E1, and the ATDC5 in the presence the hydrogels. The vertical axis in FIG. 12 indicates the cell proliferative activity (absorbance values measured). FIG. 12A shows the result of the NIH3T3. FIG. 12B shows the result of the MC3T3-E1. FIG. 12C shows the result of the ATDC5. As a result, none of the cells showed large change in cell proliferation activity between the presence and absence of the gels. In addition, the cell proliferation activity did not change even if quantity of the gels was increased. Therefore, it was revealed that the hydrogels did not show cytotoxicity for various cells. Therefore, it was shown that the hydrogels of the present invention could be used favorably as biomaterial.

### INDUSTRIAL APPLICABILITY

The present invention can be widely used in medical industry.

1. A method for manufacturing a hydrogel, the method comprising a step of mixing a first solution and a second solution to obtain a mixed solution,
wherein the first solution comprises a first four-branching compound and a first buffer solution,
wherein the second solution comprises a second four-branching compound and a second buffer solution,
wherein the first four-branching compound is shown as following formula (I), 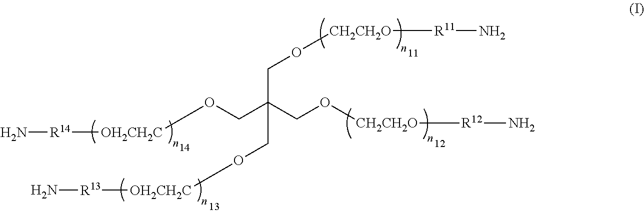
(in the formula (I), n₁₁ to n₁₄ are, each may be the same or different, an integer that is any one of 25 to 250,
in the formula (I), R¹¹ to R¹⁴ are, each may be the same or different, C₁-C₇ alkylene group, C₂-C₇ alkenylene group, —NH—R¹⁵—, —CO—R¹⁵—, —R¹⁶—O—R¹⁷—, —R¹⁶—NH—R¹⁷—, —R¹⁶—CO₂—R¹⁷—, —R¹⁶—CO₂—NH—R¹⁷—, —R¹⁶—CO—R¹⁷—, or —R¹⁶—CO—NH—R¹⁷—,
wherein R¹⁵ is C₁-C₇ alkylene group, R¹⁶ is C₁-C₃ alkylene group, and R¹⁷ is C₁-C₅ alkylene group)
wherein the second four-branching compound is shown as following formula (II), 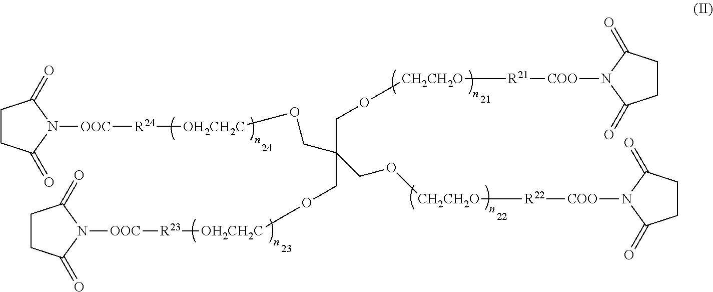
(In the formula (II), n₂₁ to n₂₄ are, each may be the same or different, an integer that is any one of 20 to 250,
in the formula (II), R²¹ to R²⁴ are, each may be the same or different, C₁-C₇ alkylene group, C₂-C₇ alkenylene group, —NH—R²⁵—, —CO—R²⁵—, —R²⁶—O—R²⁷—, —R²⁶—NH—R²⁷—, —R²⁶—CO₂—R²⁷—, —R²⁶—CO₂—NH—R¹⁷—, —R²⁶—CO—R²⁷, or —R²⁶—CO—NH—R²⁷,
wherein R²⁵ is C₁-C₇ alkylene group, R²⁶ is C₁-C₃ alkylene group, R²⁷ is C₁-C₅ alkylene group),
wherein pH of the first solution is higher than pH of the second solution,
wherein pH of the first buffer solution is from 5 to 9 and the concentration of the first buffer solution is from 20 to 200 mM, and
wherein pH of the second buffer solution is from 5 to 9 and the concentration of the first buffer solution is from 20 to 200 mM.

2. The method in accordance with claim 1, wherein the R¹¹ to R¹⁴ is C₁-C₇ alkylene group,
wherein the R²¹ to R²⁴ is —CO—R²⁵— and R²⁵ is C₁-C₇ alkylene group.

3. The method in accordance with claim 1, wherein the R¹¹ to R¹⁴ is C₂-C₄ alkylene group,
wherein R²¹ to R²⁴ is —CO—R²⁵— and R²⁵ is C₂-C₄ alkylene group.

4. The method in accordance with claim 1, wherein the first buffer solution comprises one or both of phosphate buffer and phosphate buffered saline, and
wherein the second buffer solution comprises one or more of phosphate buffer, citric acid.phosphate buffer, phosphate buffered saline, and citric acid.phosphate buffered saline.

5. The method in accordance with claim 1, wherein salt concentration of the mixed solution is 1×10⁻¹ to 1×10² mM.

6. The method in accordance with claim 1, wherein the first buffer solution is 20 mM to 100 mM phosphate buffer and pH of the first buffer solution is 5 to 9,
wherein the second buffer solution is 20 mM to 100 mM phosphate buffer and pH of the first buffer solution is 5 to 7.5, or 20 mM to 100 mM citric acid/phosphate buffer and pH of the first buffer solution is 5 to 7.5.

7. A hydrogel that is manufactured by a method which comprises a step of mixing a first solution and a second solution to obtain a mixed solution,
wherein the first solution comprises a first four-branching compound and a first buffer solution,
wherein the second solution comprises a second four-branching compound and a second buffer solution,
wherein the first four-branching compound is shown as following formula (I), 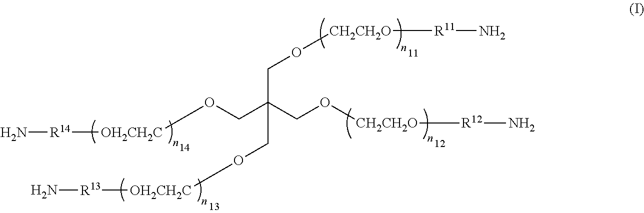
(in the formula (I):
n₁₁ to n₁₄ are, each may be the same or different, an integer that is any one of 25 to 250,
in the formula (I), R¹¹ to R¹⁴ are, each may be the same or different, C₁-C₇ alkylene group, C₂-C₇ alkenylene group, —NH—R¹⁵—, —CO—R¹⁵—, —R¹⁶—O—R¹⁷—, —R¹⁶—NH—R¹⁷—, —R¹⁶—CO₂—R¹⁷—, —R¹⁶—CO₂—NH—R¹⁷—, —R¹⁶—CO—R¹⁷—, or —R¹⁶—CO—NH—R¹⁷—,
wherein R¹⁵ is C₁-C₇ alkylene group, R¹⁶ is C₁-C₃ alkylene group, and R¹⁷ is C₁-0₅ alkylene group)
wherein the second four-branching compound is shown as following formula (II), 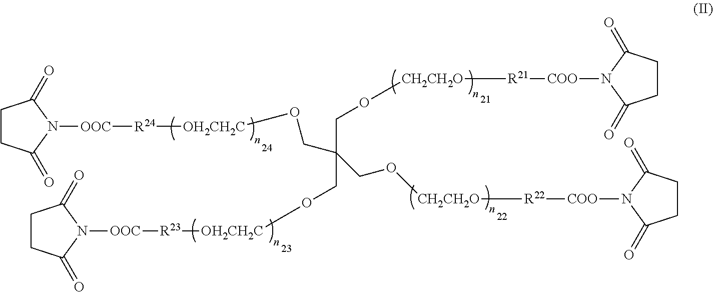
(In the formula (II), n₂₁ to n₂₄ are, each may be the same or different, an integer that is any one of 20 to 250,
in the formula (II), R²¹ to R²⁴ are, each may be the same or different, C₁-C₇ alkylene group, C₂-C₇ alkenylene group, —NH—R²⁵—, —CO—R²⁵—, —R²⁶—O—R²⁷—, —R²⁶—NH—R²⁷—, —R²⁶—CO₂—R²⁷—, —R²⁶—CO₂—NH—R¹⁷—, —R²⁶—CO—R²⁷—, or —R²⁶—CO—NH—R²⁷,
wherein R²⁵ is C₁-C₇ alkylene group, R²⁶ is C₁-C₃ alkylene group, R²⁷ is C₁-C₅
alkylene group),
wherein pH of the first solution is higher than pH of the second solution,
wherein pH of the first buffer solution is from 5 to 9 and the concentration of the first buffer solution is from 20 to 200 mM, and
wherein pH of the second buffer solution is from 5 to 9 and the concentration of the first buffer solution is from 20 to 200 mM.

8. The hydrogel in accordance with claim 7, wherein the hydrogel comprises the first four-branching compound and the second four-branching compound, wherein the composition ratio of the first four-branching compound and the second four-branching compound is 0.8:1-1.2:1,
wherein the first four-branching compound is shown as following formula (I), 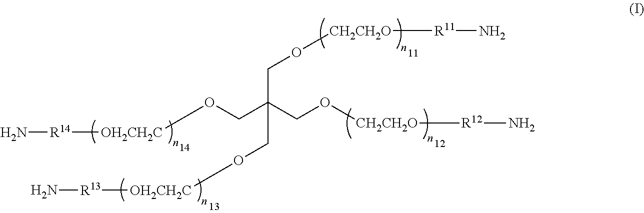
(in the formula (I), n₁₁ to n₁₄ are, each may be the same or different, an integer that is any one of 25 to 250,
in the formula (I), R¹¹ to R¹⁴ are, each may be the same or different, C₁-C₇ alkylene group, C₂-C₇ alkenylene group, —NH—R¹⁵—, —CO—R¹⁵—, —R¹⁶—O—R¹⁷—, —R¹⁶—NH—R¹⁷—, —R¹⁶—CO₂—R¹⁷—, —R¹⁶—CO₂—NH—R¹⁷—, —R¹⁶—CO—R¹⁷—, or —R¹⁶—CO—NH—R¹⁷—,
wherein R¹⁵ is C₁-C₇ alkylene group, R¹⁶ is C₁-C₃ alkylene group, and R¹⁷ is C₁-C₅ alkylene group)
wherein the second four-branching compound is shown as following formula (II), 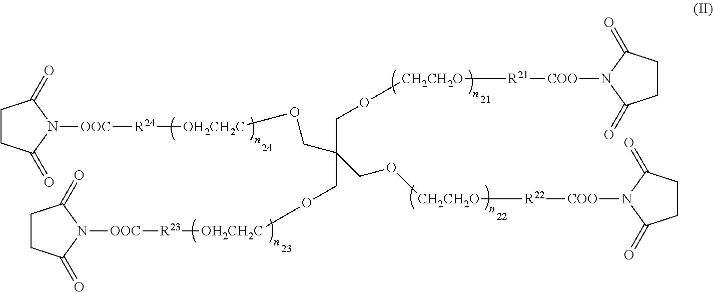
(In the formula (II), n₂₁ to n₂₄ are, each may be the same or different, an integer that is any one of 20 to 250,
R²¹ to R²⁴ are, each may be the same or different, C₁-C₇ alkylene group, C₂-C₇ alkenylene group, —NH—R²⁵—, —CO—R²⁵—, —R²⁶—O—R²⁷—, —R²⁶—NH—R²⁷—, —R²⁶—CO₂—R²⁷—, —R²⁶—CO₂—NH—R¹⁷—, —R²⁶—CO—R²⁷—, or —R²⁶—CO—NH—R²⁷,
wherein R²⁵ is C₁-C₇ alkylene group, R²⁶ is C₁-C₃ alkylene group, R²⁷ is C₁-C₅ alkylene group),
wherein the neutron scattering curve of the hydrogel can be fitted by Orstein-Zernike function.

9. The hydrogel in accordance with claim 8, wherein compressive breaking strength of the hydrogel is 10 to 120 MPa

10. A hydrogel which comprises a first four-branching compound, a second four-branching compound and a third four-branching compound, wherein the composition ratio of the first four-branching compound a second four-branching compound and a third four-branching compound is 0.3-0.7:0.1-0.65:0.1-0.65,
wherein the first four-branching compound is shown as the following formula (I), 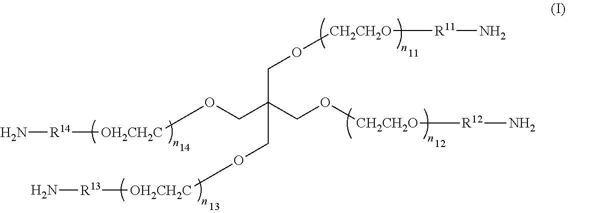
(in the formula (I), n₁₁ to n₁₄ are, each may be the same or different, an integer that is any one of 50 to 60, R¹¹ to R¹⁴ are, each may be the same or different, C₁-C₇ alkylene group)
wherein the second four-branching compound is shown as the following formula (II), 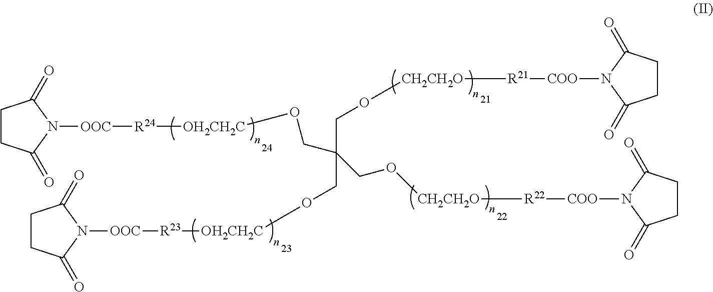
(in the formula (II), n₂₁ to n₂₄ are, each may be the same or different, an integer that is any one of 45 to 55, R²¹ to R²⁴ are, each may be the same or different, —CO—R²⁵— and R²⁵ is C₁-C₇ alkylene group.)
wherein the third four-branching compound is shown as the formula (II),
(in the formula (II), n₂₁ to n₂₄ are, each may be the same or different, an integer that is any one of 45 to 55, R²¹ to R²⁴ are, each may be the same or different, C₁-C₇ alkylene group)
